(19) United States
(12) Patent Application Publication
(10) Pub. No.: US 2012/0107248 A1
(43) Pub. Date: May 3, 2012

(54) IMAGING GASTROINTESTINAL VOLUMES AND MOTILITY

(75) Inventors: Adil E. Bharucha, Rochester, MN (US); Abdul H. Fauq, Jacksonville, FL (US)

(73) Assignee: MAYO FOUNDATION FOR MEDICAL EDUCATION AND RESEARCH, Rochester, MN (US)

(21) Appl. No.: 13/382,696

(22) PCT Filed: Jun. 28, 2010
(86) PCT No.: PCT/US10/40181

§ 371 Date: Jan. 6, 2012

Related U.S. Application Data
(60) Provisional application No. 61/223,771, filed on Jul. 8, 2009.

(51) Int. Cl. A61K 49/10; C07F 5/00

(52) U.S. Cl. 424/9.363; 534/16

(57) ABSTRACT

This disclosure relates to contrast agents and compositions comprising the same that are capable of blocking the hydrogen/potassium adenosine triphosphatase enzyme system, and more particularly to the use of such compositions for imaging stomach and colon volume and motility.

### CROSS-REFERENCE TO RELATED APPLICATIONS

This application is claims the benefit of priority to U.S. Provisional Application Serial No. 61/223,771, filed on Jul. 8, 2009.

### TECHNICAL FIELD

This disclosure relates to contrast agents and compositions comprising the same, and more particularly to the use of such compositions for MR imaging, including MR imaging of the stomach and colon and gastrointestinal parameters such as gastric (e.g., stomach) and colonic volume, motility, and transit.

### BACKGROUND

Diagnostic imaging techniques, such as magnetic resonance imaging (MRI), have been used in medical diagnosis for a number of years. The addition of contrast media has improved or increased the resolution of the image or has provided specific diagnostic information.

Many common conditions affecting the stomach (e.g., indigestion (“dyspepsia”), gastroparesis, vomiting) or colon (e.g., constipation and diarrhea) are caused by disordered motility (i.e., impaired contraction and relaxation) which in turn, results in exaggerated or delayed transit (movement) of contents. Gastric motor functions are currently assessed by measuring (i) the time required for food to be emptied from the stomach by scintigraphy, ultrasound (US), breath tests, or MRI; (ii) stomach volumes, e.g., before and after a meal, by US, MRI, or SPECT; and (iii) stomach contractility by pressure sensors (manometry) within the stomach, MRI or US. Colonic functions are assessed by measuring the time required for contents to travel across the colon by scintigraphy or radioopaque markers. Colon contractility is measured by pressure sensors (manometry) or a balloon within the colon.

Existing techniques to assess stomach and colonic functions suffer from limitations e.g., radiation exposure, invasiveness, and limited accuracy). Imaging gastrointestinal function, including stomach and colonic volume and motility, is complex and subject to a variety of technical difficulties and practical limitations. Commercially available intravenously injected gadolinium compounds are ineffective and inefficient for labeling the stomach or colon wall because gadolinium is cleared rapidly. Thus, even after intravenous gadolinium, there is not sufficient signal to demarcate the stomach or colon wall from surrounding tissues. In addition, to image both stomach volume and motility using standard MRI contrast agents can require multiple scans and multiple injections of a contrast agent. Therefore, defining the stomach contour, which is necessary to assess its volume or contractility, is a cumbersome, manual, and time-consuming process limited to research studies only. Given an appropriate targeted contrast agent, however, the non-invasive nature of MRI imaging could allow for improved study of gastrointestinal function and structure.

### SUMMARY

Contrast agents and compositions comprising the same and methods of imaging using such agents and compositions are described. The contrast agents described herein incorporate one or more proton pump inhibitor targeting moieties. For example, a contrast agent, as described herein can include a compound of formula I:

[PPI]ₙ−[L]ₘ−[C]ₚ

Wherein PPI is a proton pump inhibitor; L is a linker; C is a physiologically compatible metal chelating group; n is an integer from one to five; m is an integer from zero to ten; and p is an integer from one to ten; or a pharmaceutically acceptable salt thereof.

In some embodiments, the PPI is chosen from omeprazole, lansoprazole, dexlansoprazole, esomeprazole, pantoprazole, and rabeprazole. In some embodiments, the PPI comprises a compound of formula II:

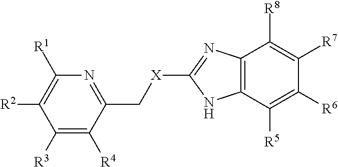

wherein X is S or S═O; R¹, R², R³, R⁴, R⁵, R⁶, R⁷, and R⁸ are independently selected from H, C₁₋₁₀ alkyl, C₂₋₁₀ alkenyl, C₂₋₁₀ alkynyl, or OR⁹, NR⁹, or SR⁹, wherein at least one of R¹-R⁸ is OR⁹; R⁹ is independently selected from H, C₁₋₁₀ alkyl, C₂₋₁₀ alkenyl, C₂₋₁₀ alkynyl, or a linkage site to L, if present, or C, wherein at least one R⁹ is a linkage site; or a pharmaceutically acceptable salt thereof. In some embodiments, the compound of formula II can be:

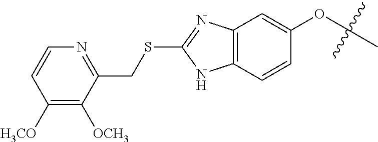

or a pharmaceutically acceptable salt thereof. In some embodiments, the compound of formula II can be:

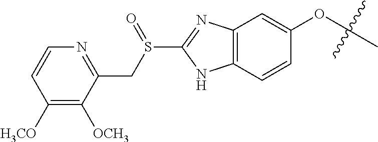

or a pharmaceutically acceptable salt thereof.

The physiologically compatible metal chelating group (C) can be complexed to a paramagnetic metal ion. In some embodiments, the paramagnetic metal ion is chosen from Gd(III), Fe(III), Mn(II), Mn(III), Cr(III), Cu(II), Dy(III), Ho(III), Er(III), Pr(III), Eu(II), Eu(III), Tb(III), and Tb(IV). In some embodiments, the paramagnetic metal ion is Gd(III). The physiologically compatible metal chelating group (C) can include a cyclic or an acyclic organic chelating agent. In some embodiments, the cyclic or acyclic organic chelating agent is chosen from DTPA, DOTA, HP-DO3A, NOTA, DOTAGA, Glu-DTPA, and DTPA-BMA. In some embodiments, the cyclic or acyclic organic chelating agent comprises DTPA, DOTAGA, and DOTA.

In some embodiments, the compound of formula I is:

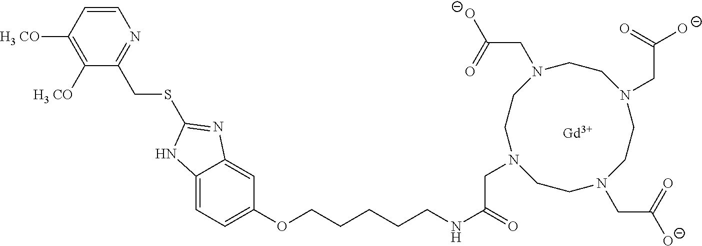

or a pharmaceutically acceptable salt thereof. In some embodiments, the compound of formula I is:

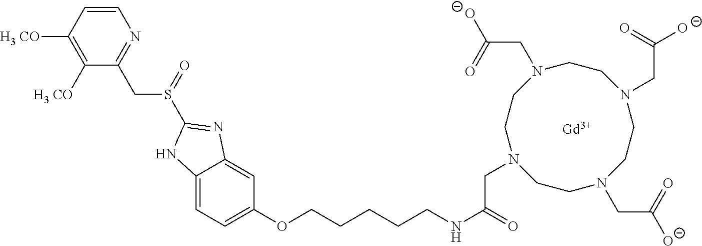

or a pharmaceutically acceptable salt thereof.

Further provided herein is a pharmaceutical composition comprising a contrast agent having a compound of formula I or pharmaceutically acceptable salt thereof, as provided herein, and a pharmaceutically acceptable carrier, adjuvant or vehicle.

Methods of using a contrast agent comprising a compound of formula I are also provided herein. For example, a method of MRI imaging is provided, the method includes administering to a subject an effective amount of a contrast agent comprising a compound of formula I, or pharmaceutically acceptable salt thereof, and performing MRI imaging on the subject.

Also provided are methods of imaging the stomach of a subject. The method can include administering to the subject an effective amount of a contrast agent comprising a compound of formula I, or pharmaceutically acceptable salt thereof, and performing MRI imaging of the stomach. In some embodiments, the imaging of the stomach comprises imaging of the stomach wall. In some embodiments, the imaging of the stomach comprises imaging of the stomach contents. In some embodiments, the imaging of the stomach comprises imaging of the stomach wall and contents simultaneously.

This disclosure also provides a method of imaging the stomach wall of a subject. The method includes administering to the subject an effective amount of a contrast agent comprising a compound of formula I, or pharmaceutically acceptable salt thereof, and performing MRI imaging of the stomach wall. Also provided is a method of imaging the stomach contents of a subject. The method can include administering to the subject an effective amount of a contrast agent comprising a compound of formula I, or pharmaceutically acceptable salt thereof, and performing MRI imaging of the stomach contents.

Further provided herein is a method of imaging the stomach wall and stomach contents of a subject simultaneously, the method includes administering to the subject an effective amount of a contrast agent comprising a compound of formula I, or pharmaceutically acceptable salt thereof, and performing MRI imaging of the stomach wall and stomach contents.

Also provided herein is a method of imaging stomach volume of a subject, the method includes administering to the subject an effective amount of a contrast agent comprising a compound of formula I, or pharmaceutically acceptable salt thereof, and performing MRI imaging of the stomach contents.

The contrast agents as described herein can also be utilized to image stomach motility of a subject. The method can include administering to the subject an effective amount of a contrast agent comprising a compound of formula I, or pharmaceutically acceptable salt thereof, and performing MRI imaging of the stomach wall. The method can also be used to assess stomach emptying of a subject. The method can include administering to the subject an effective amount of a contrast agent comprising a compound of formula I, or pharmaceutically acceptable salt thereof, and performing MRI imaging of the stomach wall. The stomach contents may be visualized simultaneously and its emptying measured over time.

Also described herein is a method of imaging stomach volume and motility of a subject, the method includes administering to the subject an effective amount of a contrast agent comprising a compound of formula I, or pharmaceutically acceptable salt thereof, and performing MRI imaging of the stomach. In some embodiments, the imaging of the stomach comprises imaging of the stomach wall. In some embodiments, the imaging of the stomach comprises imaging of the stomach contents. In some embodiments, the imaging of the stomach comprises imaging of the stomach wall and contents simultaneously. In some embodiments, the MRI imaging of the stomach is performed using a single MRI sequence. In some embodiments, the subject is a human.

Also provided are methods of imaging the colon of a subject. The method can include administering to the subject an effective amount of a contrast agent comprising a compound of formula I, or pharmaceutically acceptable salt thereof, and performing MRI imaging of the colon. In some embodiments, the imaging of the colon comprises imaging of the colon wall. In some embodiments, the imaging of the colon comprises imaging of the colon contents. In some embodiments, the imaging of the colon comprises imaging of the colon wall and contents simultaneously.

Further provided herein is a method of imaging viscera responsive to proton pump inhibitors in a subject. The method includes administering to the subject an effective amount of a contrast agent comprising a compound of formula I, or pharmaceutically acceptable salt thereof, and performing MRI imaging of the viscera. In some embodiments, the imaging of the viscera comprises imaging the wall of the viscera. In some embodiments, the imaging of the viscera comprises imaging of the contents of the viscera. In some embodiments, the imaging of the viscera comprises imaging of the wall and the contents of the viscera simultaneously. In some embodiments, the viscera is selected from one or more of the stomach, colon, kidneys, intestine, liver, and bladder.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1 illustrates the observed isotopic distribution of a pantoprazole-DOTA-gadolinium conjugate by HPLC.

FIG. 2 is a comparison of images showing gastric configuration without administration of intravenous gadolinium under fasting conditions.

FIG. 3 is a comparison of gastric configuration images after intravenous administration of Magnevist (Study 1) and a PPI-gadolinium complex, as described herein, (Study 2) under fasting conditions.

FIG. 4 is a comparison of gastric configuration images after intravenous administration of Magnevist (Study 1) and a PPI-gadolinium complex, as described herein, (Study 2) under postprandial conditions.

FIG. 5 is a comparison of bladder appearance images after intravenous administration of Magnevist (Study 1) and a PPI-gadolinium complex, as described herein (Studies 2 and 3) under postprandial conditions.

FIG. 6 illustrates the increase in gastric wall signal and automated segmentation in gastric MRI after imaging with a PPI-gadolinium complex compared to Magnevist alone. The arrow shows increased signal outside the stomach after Magnevist. The segmentation program misidentified the gastric wall (i.e., thick line) to be outside the stomach after Magnevist but accurately identified the wall after the PPI-gadolinium complex.

FIG. 7 compares the splenic uptake with Magnevist and a PPI-gadolinium complex. Arrow shows the spleen. The thick and thin lines depict the gastric wall and lumen respectively as identified by the segmentation program.

FIG. 8 illustrates the accuracy of gastric wall thickness measurements by Magnevist and a PPI-gadolinium complex.

FIG. 9 shows the increased signal in gastric wall can be sustained for a longer during after administration of a PPI-gadolinium complex compared to Magnevist.

FIG. 10a compares the colonic wall signal following administration of Magnevist (left panel) and a PPI-gadolinium complex (right panel), FIG. 10b shows the postprandial post contrast colonic wall images for the two contrast agents.

### DETAILED DESCRIPTION

Contrast agents and compositions comprising the same and methods of imaging using such agents and compositions are described. The contrast agents described herein incorporate one or more proton pump inhibitor targeting moieties. In some embodiments, the incorporation of a proton pump inhibitor targeting moiety allows the contrast agent to be capable of blocking the hydrogen/potassium adenosine triphosphatase enzyme system. Accordingly, this moiety can target the contrast agent to organs responsive to proton pump inhibitors. For example, the contrast agents can bind to both the stomach and colonic wall and its contents, facilitating simultaneous measurement of stomach and colonic volume and motility.

### Definitions

Unless defined otherwise, all technical and scientific terms used herein have the same meaning as is commonly understood by one of ordinary skill in the art to which this disclosure belongs. All patents, applications, published applications, and other publications are incorporated by reference in their entirety. In the event that there is a plurality of definitions for a term herein, those in this section prevail unless stated otherwise.

As used herein, “administration” refers to delivery of a contrast agent by any external route, including, without limitation, IV, intramuscular, SC, intranasal, inhalation, transdermal, oral, rectal, sublingual, and parenteral administration.

The expression “effective amount,” when used to describe an amount of contrast agent administered in a method, refers to the amount of a contrast agent that achieves the desired pharmacological or imaging effect.

As used herein, “subject” (as in the subject of the treatment) means both mammals and non-mammals. Mammals include, for example, humans; non-human primates, e.g. apes and monkeys; cattle; horses; sheep; rats; mice; pigs; and goats. Non-mammals include, for example, fish and birds.

Commonly used chemical abbreviations that are not explicitly defined in this disclosure may be found in The American Chemical Society Style Guide, Second Edition; American Chemical Society, Washington, D.C. (1997) and “2001 Guidelines for Authors” J. Org. Chem. 66(1), 24A (2001).

The term “alkyl” includes saturated aliphatic groups, including straight-chain alkyl groups (e.g., methyl, ethyl, propyl, butyl, pentyl, hexyl, heptyl, octyl, nonyl, and decyl) and branched-chain alkyl groups (e.g, isopropyl, tert-butyl, and isobutyl). The term alkyl further includes alkyl groups, which can further include oxygen, nitrogen, sulfur or phosphorous atoms replacing one or more carbons of the hydrocarbon backbone. In certain embodiments, a straight chain or branched chain alkyl has 6 or fewer carbon atoms in its backbone (e.g., C₁-C₆ for straight chain, C₃-C₆ for branched chain), and more preferably 4 or fewer. The term C₁-C₆ includes alkyl groups containing 1 to 6 carbon atoms.

Moreover, the term “alkyl” includes both “unsubstituted alkyls” and “substituted alkyls,” the latter of which refers to alkyl moieties having substituents replacing a hydrogen on one or more carbons of the hydrocarbon backbone. Such substituents can include, for example, alkyl, alkenyl, alkynyl, halogen, hydroxyl, carboxylate, alkoxyl, amino (including alkyl amino, dialkylamino, arylamino, diarylamino, and alkylarylamino), trifluoromethyl, alkylaryl, or an aromatic moiety. The term “n-alkyl” means a straight chain (i.e., unbranched) unsubstituted alkyl group.

The term “alkenyl” includes aliphatic groups that may or may not be substituted, as described above for alkyls, containing at least one double bond and at least two carbon atoms. For example, the term “alkenyl” includes straight-chain alkenyl groups (e.g., ethylenyl, propenyl, butenyl, pentenyl, hexenyl, heptenyl, octenyl, nonenyl, and decenyl) and branched-chain alkenyl groups. The term alkenyl further includes alkenyl groups that include oxygen, nitrogen, sulfur or phosphorous atoms replacing one or more carbons of the hydrocarbon backbone. In certain embodiments, a straight chain or branched chain alkenyl group has 6 or fewer carbon atoms in its backbone (e.g., C₂-C₆ for straight chain, C₃-C₆ for branched chain). The term C₂-C₆ includes alkenyl groups containing 2 to 6 carbon atoms.

Moreover, the term “alkenyl” includes both “unsubstituted alkenyls” and “substituted alkenyls,” the latter of which refers to alkenyl moieties having substituents replacing a hydrogen on one or more carbons of the hydrocarbon backbone. Such substituents can include, for example, alkyl, alkenyl, alkynyl, halogen, hydroxyl, carboxylate, alkoxyl, amino (including alkyl amino, dialkylamino, arylamino, diarylamino, and alkylarylamino), trifluoromethyl, alkylaryl, or an aromatic moiety.

The term “alkynyl” includes unsaturated aliphatic groups analogous in length and possible substitution to the alkyls described above, but which contain at least one triple bond and two carbon atoms. For example, the term “alkynyl” includes straight-chain alkynyl groups (e.g., ethynyl, propynyl, butynyl, pentynyl, hexynyl, heptynyl, octynyl, nonynyl, and decynyl) and branched-chain alkynyl groups. The term alkynyl further includes alkynyl groups that include oxygen, nitrogen, sulfur or phosphorous atoms replacing one or more carbons of the hydrocarbon backbone. In certain embodiments, a straight chain or branched chain alkynyl group has 6 or fewer carbon atoms in its backbone (e.g., C₂-C₆ for straight chain, C₃-C₆ for branched chain). The term C₂-C₆ includes alkynyl groups containing 2 to 6 carbon atoms.

For the purposes of this application, “DTPA” refers to a chemical compound comprising a substructure composed of diethylenetriamine, wherein the two primary amines are each covalently attached to two acetyl groups and the secondary amine has one acetyl group covalently attached according to the following formula:

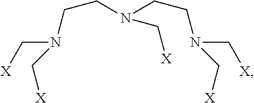

wherein each X is independently a functional group capable of coordinating a metal cation, preferably COO⁻, COOH, C(O)NH₂, C(O)NHR, C(O)NRR′, PO₃²⁻, PO₃R⁻, P(R)O₂⁻ or NHR, or OR wherein R is any aliphatic group. When each X group is the tert-butoxy (^{t}Bu) carboxylate ester (COO^{t}Bu), the structure may be referred to as “DTPE” (“E” for ester).

For the purposes of this application, “DOTA” refers to a chemical compound comprising a substructure composed of 1,4,7,11-tetraazacyclododecane, wherein the amines each have one acetyl group covalently attached according to the following formula:

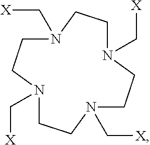

wherein X is defined above.

For the purposes of this application, “NOTA” refers to a chemical compound comprising a substructure composed of 1,4,7-triazacyclononane, wherein the amines each have one acetyl group covalently attached according to the following formula:

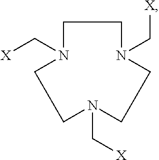

wherein X is defined above.

For the purposes of this application, “DO3A” refers to a chemical compound comprising a substructure composed of 1,4,7,11-tetraazacyclododecane, wherein three of the four amines each have one acetyl group covalently attached and the other amine has a substituent having neutral charge according to the following formula:

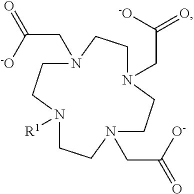

wherein R¹ is an uncharged chemical moiety, preferably hydrogen, any aliphatic group and uncharged derivatives thereof. The chelate “HP”-DO3A has R¹═—CH₂(CHOH)CH₃.

The terms “chelating ligand,” “chelating moiety,” and “chelate moiety” may be used to refer to any polydentate ligand which is capable of coordinating a metal ion, including DTPA (and DTPE), DOTA, DO3A, DOTAGA, Glu-DTPA, or NOTA molecule, or any other suitable polydentate chelating ligand as is further defined herein, that is either coordinating a metal ion or is capable of doing so, either directly or after removal of protecting groups. The term “chelate” refers to the actual metal-ligand complex, and it is understood that the polydentate ligand will eventually be coordinated to a medically useful metal ion.

### Contrast Agents

A contrast agent can include a compound of formula I:

[PPI]ₙ−[L]ₘ−[C]ₚ

wherein PPI is a proton pump inhibitor, L is a linker, C is a physiologically compatible metal chelating group, n is an integer from one to five (e.g., 1, 2, 3, 4, and 5); m is an integer from zero to ten (e.g., 0, 1, 2, 3, 4, 5, 6, 7, 8, 9, and 10); and p is an integer from one to ten (e.g., 1, 2, 3, 4, 5, 6, 7, 8, 9, and 10); or a pharmaceutically acceptable salt thereof.

A proton pump inhibitor (PPI) can be any compound that acts by blocking the hydrogen/potassium adenosine triphosphatase enzyme system. Examples of proton pump inhibitors include omeprazole, lansoprazole, dexlansoprazole, esomeprazole, pantoprazole, rabeprazole, or potassium-competitive acid blockers (e.g., soraprazan and revaprazan). In some embodiments, the proton pump inhibitor is pantoprazole. A PPI can also include any compound which functions as an H2 antagonist and acts by blocking the action of histamine on parietal cells. Examples of such compounds include cimetidine, ranitidine, famotidine, and nizatidine.

A proton pump inhibitor can also be a compound of formula II:

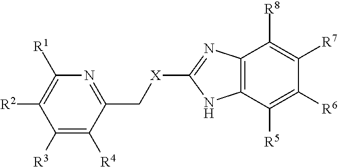

wherein X is S or S═O; R¹, R², R³, R⁴, R⁵, R⁶, R⁷, and R⁸ are independently selected from H, C₁₋₁₀ alkyl, C₂₋₁₀ alkenyl, C₂₋₁₀ alkynyl, OR⁹, NR⁹, or SR⁹, wherein at least one of R¹-R⁸ is OR⁹, NR⁹, or SR⁹; R⁹ is independently selected from H, C₁₋₁₀ alkyl, C₂₋₁₀ alkenyl, C₂₋₁₀ alkynyl, or is a linkage site to L, if present, or C, wherein at least one R⁹ is a linkage site; or a pharmaceutically acceptable salt thereof.

In some embodiments, R¹ is H. In some embodiments, R² is H or a C₁₋₁₀ alkyl. In some embodiments, R² is CH₃. In some embodiments, R³ is H or OR⁹. In some embodiments, R³ is selected from OCH₃, OCH₂CF₃, and O(CH₂)₃OCH₃. In some embodiments, R⁴ is selected from H, C₁₋₁₀ alkyl, or OR⁹. In some embodiments, R⁴ is selected from CH₃ or OCH₃. In some embodiments, R⁵ is H or OR⁹. In some embodiments, R⁵ is OCHF₂. In some embodiments, R⁶ is H or OR⁹. In some embodiments, R⁶ is OCH₃. In some embodiments, R⁷ is H or OR⁹. In some embodiments, R⁷ is OCH₃. In some embodiments, R⁸ is H. In some embodiments, R⁹ is a C₁₋₁₀ alkyl. In some embodiments, R⁹ is selected from CH₃, CHF₂, CH₂CF₃, or (CH₂)₃OCH₃.

In some embodiments, the compound of formula II can be

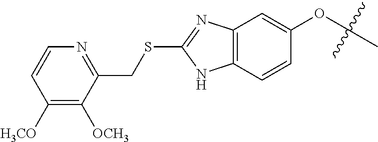

or a pharmaceutically acceptable salt thereof. In some embodiments, the compound of formula II can be

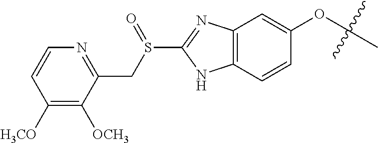

or a pharmaceutically acceptable salt thereof.

A PPI may be provided in its neutral form or in the form of an alkali metal salt, in the racemic form or in the form of a pure enantiomer, or in any polymorphic form.

A linker (L) can be any physiologically compatible chemical group that does not interfere with the functions of the proton pump inhibitor or chelating group. Preferred linkers are synthetically easy to incorporate into the contrast agent. They are also not so unduly large as to manifest an undesired biological function or targeting influence onto the contrast agent. Preferably, the length of the linker is between 1 and 50 angstroms, more preferably 1 and 10 angstroms.

In some embodiments, the linker is a C₁₋₁₀ alkyl group or an C₁₋₁₀ alkyl amine group. In some embodiments, the linker is chosen from —(—O—CHR—CHR¹⁰—O—)_{q} and (—NHCO—CHR¹⁰—CHR¹⁰—NHCO—)_{q} wherein each R¹⁰ is independently an unsubstituted or substituted C₁₋₁₀ alkyl group and q is an integer from 1-5.

A physiologically acceptable metal chelating group (C) can be any of the many known in the art, and includes, for example, cyclic and acyclic organic chelating agents such as DTPA, DOTA, HP-DO3A, DOTAGA, NOTA, Glu-DTPA, and DTPA-BMA. For MRI, metal chelates such as gadolinium diethylenetriaminepentaacetate (DTPA.Gd), gadolinium tetraamine 1,4,7,10-tetraazacyclododecane-N,N′,N″,N′″-tetraacetate (DOTA.Gd), gadolinium 1,4,7,10-tetraazacyclododecane-1,4,7-triacetate (DO3A.Gd), and bb(CO)DTPA.Gd are particularly useful. In certain embodiments, DOTAGA may be used. The structure of DOTAGA, shown complexed with Gd(III), is as follows:

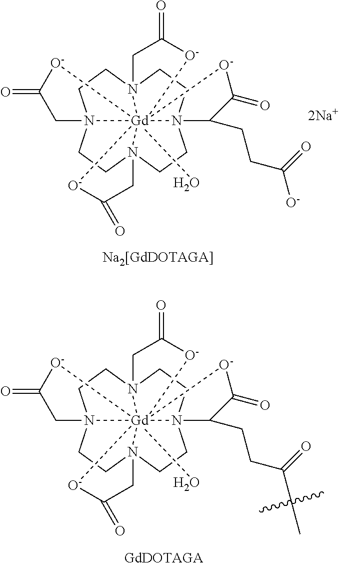

In other cases, the C can be GluDTPA, which has the following structure (shown complexed with Gd(III):

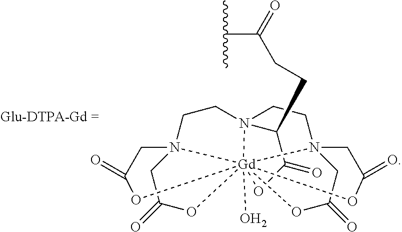

In some embodiments, C is DOTA.

For MRI applications, the chelating group can be complexed to a paramagnetic metal ion, including Gd(III), Fe(III), Mn(II), Mn(III), Cr(III), Cu(II), Dy(III), Ho(III), Er(III), Pr(III), Eu(II), Eu(III), Tb(III), Tb(IV), Tm(III), and Yb(III). In some embodiments, the paramagnetic metal ion is Gd(III). Additional information regarding chelating groups and synthetic methodologies for incorporating them into a contrast agent can be found in WO 01/09188, WO 01/08712, and U.S. Pat. No. 7,238,341.

Metal chelates should not dissociate metal to any significant degree during the contrast agent's passage through the body, including while bound to a target.

In some embodiments, a contrast agent as provided herein can be chosen from:

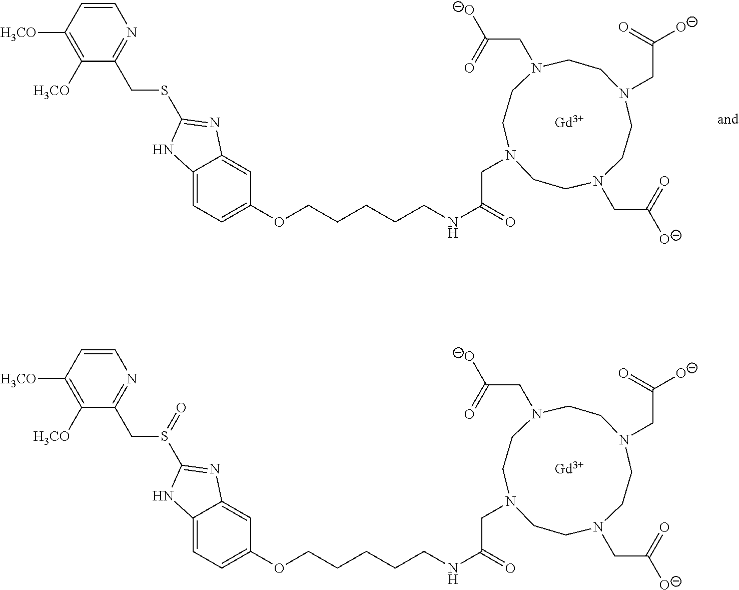

or a pharmaceutically acceptable salt thereof.

Contrast agents as described herein can be synthesized using standard organic synthesis techniques known to those of ordinary skill in the art (see Examples 1 and 2).

### Pharmaceutical Compositions and Administration

The contrast agents described herein may comprise a pharmaceutically acceptable salt. Pharmaceutically acceptable salts of this invention include those derived from inorganic or organic acids and bases. Included among such acid salts are the following: acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxy-ethanesulfonate, lactate, maleate, methanesulfonate, 2-naphthalenesulfonate, nicotinate, oxalate, pamoate, pectinate, persulfate, 3-phenyl-propionate, picrate, pivalate, propionate, succinate, tartrate, thiocyanate, tosylate and undecanoate. Base salts include ammonium salts, alkali metal salts, such as sodium and potassium salts, alkaline earth metal salts, such as calcium, magnesium and zinc salts, salts with organic bases, such as dicyclohexylamine salts, N-methyl-D-glucamine, and salts with amino acids such as arginine, lysine, and so forth. Also, the basic nitrogen-containing groups can be quaternized with such agents as lower alkyl halides, such as methyl, ethyl, propyl, and butyl chloride, bromides and iodides; dialkyl sulfates, such as dimethyl, diethyl, dibutyl and diamyl sulfates, long chain halides such as decyl, lauryl, myristyl and stearyl chlorides, bromides and iodides, aralkyl halides, such as benzyl and phenethyl bromides and others. Water or oil-soluble or dispersible products are thereby obtained. The preferred salts of this invention are the N-methyl-D-glucamine, calcium and sodium salts.

The pharmaceutical compositions of this invention comprise any of the contrast agents described herein, or pharmaceutically acceptable salts thereof, together with any pharmaceutically acceptable carrier, adjuvant or vehicle. Pharmaceutically acceptable carriers, adjuvants and vehicles that may be used in the pharmaceutical compositions described herein include, but are not limited to, ion exchangers, alumina, aluminum stearate, lecithin, serum proteins, such as human serum albumin, buffer substances such as phosphates, glycine, sorbic acid, potassium sorbate, TRIS (tris(hydroxymethyl)aminomethane), partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat.

According to this disclosure, the pharmaceutical compositions may be in the form of a sterile injectable preparation, for example a sterile injectable aqueous or oleaginous suspension. This suspension may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as Ph. Helv or similar alcohol.

The contrast agents and pharmaceutical compositions described herein may be administered orally, parenterally, by inhalation spray, topically, rectally, nasally, buccally, vaginally or via an implanted reservoir in dosage formulations containing conventional non-toxic; pharmaceutically-acceptable carriers, adjuvants and vehicles. The term “parenteral” as used herein includes subcutaneous, intravenous, intramuscular, intra-articular, intra-synovial, intrasternal, intrathecal, intrahepatic, intralesional and intracranial injection or infusion techniques.

When administered orally, the pharmaceutical compositions of this invention may be administered in any orally acceptable dosage form including, but not limited to, capsules, tablets, aqueous suspensions or solutions. In the case of tablets for oral use, carriers which are commonly used include lactose and corn starch. Lubricating agents, such as magnesium stearate, are also typically added. For oral administration in a capsule form, useful diluents include lactose and dried corn starch. When aqueous suspensions are required for oral use, the active ingredient is combined with emulsifying and suspending agents. If desired, certain sweetening, flavoring or coloring agents may also be added.

Alternatively, when administered in the form of suppositories for rectal administration, the pharmaceutical compositions of this invention may be prepared by mixing the agent with a suitable non-irritating excipient which is solid at room temperature but liquid at rectal temperature and therefore will melt in the rectum to release the drug. Such materials include cocoa butter, beeswax and polyethylene glycols.

As noted before, the pharmaceutical compositions of this invention may also be administered topically, especially when the target of treatment includes areas or organs readily accessible by topical application, including the skin or the lower intestinal tract. Suitable topical formulations are readily prepared for each of these areas or organs.

Topical application for the lower intestinal tract can be effected in a rectal suppository formulation (see above) or in a suitable enema formulation. Topically-transdermal patches may also be used.

For topical applications, the pharmaceutical compositions maybe formulated in a suitable ointment containing the active component suspended or dissolved in one or more carriers. Carriers for topical administration of the compounds of this invention include, but are not limited to, mineral oil, liquid petrolatum, white petrolatum, propylene glycol, polyoxyethylene, polyoxypropylene compound, emulsifying wax and water. Alternatively, the pharmaceutical compositions can be formulated in a suitable lotion or cream containing the active components suspended or dissolved in one or more pharmaceutically acceptable carriers. Suitable carriers include, but are not limited to, mineral oil, sorbitan monostearate, polysorbate 60, cetyl esters wax, cetearyl alcohol, 2-octyldodecanol, benzyl alcohol and water.

For administration by nasal aerosol or inhalation, the pharmaceutical compositions of this invention are prepared according to techniques well-known in the art of pharmaceutical formulation and may be prepared as solutions in saline, employing benzyl alcohol or other suitable preservatives, absorption promoters to enhance bioavailability, fluorocarbons, and/or other conventional solubilizing or dispersing agents.

Dosage depends on the sensitivity of the diagnostic imaging instrumentation, as well as the composition of the contrast agent. For example, for MRI imaging, a contrast agent containing a highly paramagnetic substance, e.g., gadolinium (III), generally requires a lower dosage than a contrast agent containing a paramagnetic substance with a lower magnetic moment, e.g., iron (III). Preferably, dosage will be in the range of about 0.001 to 1 mmol/kg body weight per day of the active metal-chelate-complex. More preferably, dosage will be in the range of about 0.005 and about 0.05 mmol/kg body weight per day.

It should be understood, however, that a specific dosage regimen for any particular subject will also depend upon a variety of factors, including the age, body weight, general health, sex, diet, time of administration, rate of excretion, drug combination, and the judgment of the treating physician.

### Methods of Use

Contrast agents prepared according to the disclosure herein may be used in the same manner as conventional MRI contrast agents. Typically, the contrast agent is administered to a subject (e.g., a human) and an MRI image of the subject is acquired. In some embodiments, the agents can be used to image locations within the body which are responsive to proton pump inhibitors (e.g., the stomach and colon). Accordingly, the clinician can acquire an image of an area responsive to a proton pump inhibitor and that is targeted by the agent. For example, the clinician may acquire an image of the stomach, kidneys, intestine, colon, liver, or bladder. The clinician may acquire one or more images at a time before, during, or after administration of the contrast agent. Scans may be spaced out over time, for example, one scan can be acquired followed by additional scans occurring anywhere from 1 second to 24 hours following administration (e.g., 5 seconds, 10 seconds, 15 seconds, 30 seconds, minute, 5 minutes, 15 minutes, 30 minutes, hour, 2 hours, 4 hours, 6 hours, 10 hours, 12 hours, 18 hours, and 24 hours following administration of the contrast agent). Scans can be evenly spaced (e.g., every 15 seconds, every 30 seconds, every minute, every 5 minutes, every 15 minutes, every 30 minutes, every hour, every 2 hours, every 4 hours, every 6 hours, every 10 hours, every 12 hours, every 18 hours, and every 24 hours) or spaced at different intervals as required to obtain the information of interest.

One aspect of the present application involves magnetic resonance-based imaging techniques. The magnetic resonance imaging techniques employed in the present invention are known and are described, for example, in Kean & Smith, (1986) Magnetic Resonance Imaging: Principles and Applications, Williams and Wilkins, Baltimore, Md. Contemplated MRI techniques include, but are not limited to, nuclear magnetic resonance (“NMR”) and electronic spin resonance (“ESR”). The preferred imaging modality is NMR.

Standard equipment, conditions and techniques can be used to generate images; appropriate equipment, conditions and techniques can be determined in the course of experimental design. When in vivo MRI experiments are performed in the context of the present invention, they will be performed on a suitable NMR spectrometer. Artifacts from respiratory motion can be reduced using breath-hold methodologies or free-breathing navigator techniques.

MRI techniques specific to imaging gastrointestinal bodies and functions, including stomach volume and motility, are known to those of ordinary skill in the art. See, for example, Kunz, P. et al., Radiology 207:33-40 1998; Bilecen, D. et al., Abdom. Imaging 25:30-34 2000; Kwiatek, M. A. et al., J. Mag. Res. Imaging 24:1101-1109 2006; and Fidler, J. et al., Neurogastroenterol Motil 21:42-51 2009. In some embodiments, the volume and motility of the stomach is measured in a single scan. In some embodiments, the volume and motility of the stomach are measured in multiple scans. In some embodiments, multiple MRI sequences are used to image stomach volume and motility. In some embodiments, a single MRI sequence is used to image stomach volume and motility simultaneously.

The contrast agents described herein incorporate one or more proton pump inhibitor targeting moieties. In some embodiments, the incorporation of a proton pump inhibitor targeting moiety allows the contrast agent to be capable of blocking the hydrogen/potassium adenosine triphosphatase enzyme system or the potassium binding site of the proton pump, and can target locations within the body which are responsive to proton pump inhibitors. Such binding allows the contrast agents to target and image the structure and function of such locations. For example, the contrast agents can be used to image the stomach wall, stomach contents, colon wall or colon contents, or both using a single MRI sequence or multiple MRI sequences. Similar methods can be used at other responsive locations within the body. In some embodiments, the contrast agents can be used to image the stomach wall, stomach contents, or both using a single MRI scan or multiple MRI scans.

In addition, the binding and increased half-life of the contrast agents described herein, compared to standard MRI contrast agents, facilitates time-resolved imaging of gastrointestinal structure, motility and function. Traditional MRI agents, e.g., Gd(DTPA), have fast blood clearance times (approximately 2 minutes) and therefore require multiple injections of the contrast agent in order to create time-resolved images. Such additional injections may be limited (e.g., by the expense involved or based on concerns over the number of injections or amount of paramagnetic ion permitted over a particular amount of time). Administration of the contrast agents described herein and MRI imaging can, however, have longer residence times within the body and can provide longer time-frames for imaging with fewer injections. For example, such imaging methods can be used to image gastric or colonic motility; gastric emptying; gastric accommodation; gastroduodenal motility; gastric secretion; stomach size and volume; contractile velocity, frequency, amplitude, and coordination, phasic distal antral contraction waves (ACWs), including postprandial propagation, periodicity, geometry, and percentage occlusion. In the colon, such imaging methods can be used to image colonic motility and its parameters (velocity of propagation, frequency, amplitude) colonic emptying, size and volume. Circumstances in which these measurements may be useful includes, but is not limited to, identification and characterization of structure and function in health and disease, measuring the effects of chemical agents (e.g., hormones and drugs) on stomach or colonic functions, and to use these measurements to predict the efficacy of chemical agents on stomach and colonic functions. For example, the ability to label the stomach or colon wall will facilitate more accurate measurements of their contractility and function both at baseline and in response to a meal or stimulant agents in patients with gastroparesis or chronic constipation. These measurements may guide therapy and even the choice of therapeutic agent. At the other extreme, these techniques may enhance the ability to identify exaggerated contractility and/or emptying.

In addition to the stomach, other viscera may be imaged using the contrast agents described herein. For example, these contrast agents may be used to image the kidneys, intestine, liver, or bladder. Binding of the contrast agents can allow for imaging of the walls and/or contents of the viscera. Images of the wall and contents may be obtained simultaneously or individually. In some embodiments, the wall and contents of the viscera are imaged using a single MRI sequence. In some embodiments, the wall and contents of the viscera are imaged using multiple MRI sequences. MRI images can be obtained through single or multiple scans, as described above.

In some embodiments, the compounds described herein may have functions which may be independent of their activities related to the proton pump. For example, the compounds may function as an LXR agonist. See, e.g., Cronican, A. A. et al., Biochemical Pharmacology 79 (2010) 1310-1316.

### Kits

Also provided herein are kits. Typically, a kit includes a contrast agent having a compound of formula I. In certain embodiments, a kit can include one or more delivery systems, e.g., for a contrast agent having a compound of formula I, and directions for use of the kit (e.g., instructions for imaging a subject). In some embodiments, the kit can include a contrast agent having a compound of formula I and a label that indicates that the contents are to be administered to a subject undergoing MRI imaging. In another embodiment, the kit can include a contrast agent having a compound of formula I and a label that indicates that the contents are to be administered to a subject to image stomach volume and motility. In another embodiment, the kit can include a contrast agent having a compound of formula I and a label that indicates that the contents are to be administered to a subject to image viscera.

### EXAMPLES

### Example 1

### Preparation of monogadolinium(II) mono(2,2′,2″-(10-(2-(5-(2-((3,4-dimethoxypyridin-2-yl)methyl-thio)-1H-benzo[d]imidazol-5-yloxy)pentylamino)-2-oxoethyl)-1,4,7,10-tetraazacyclo-dodecane-1,4,7-triyl)triacetate)

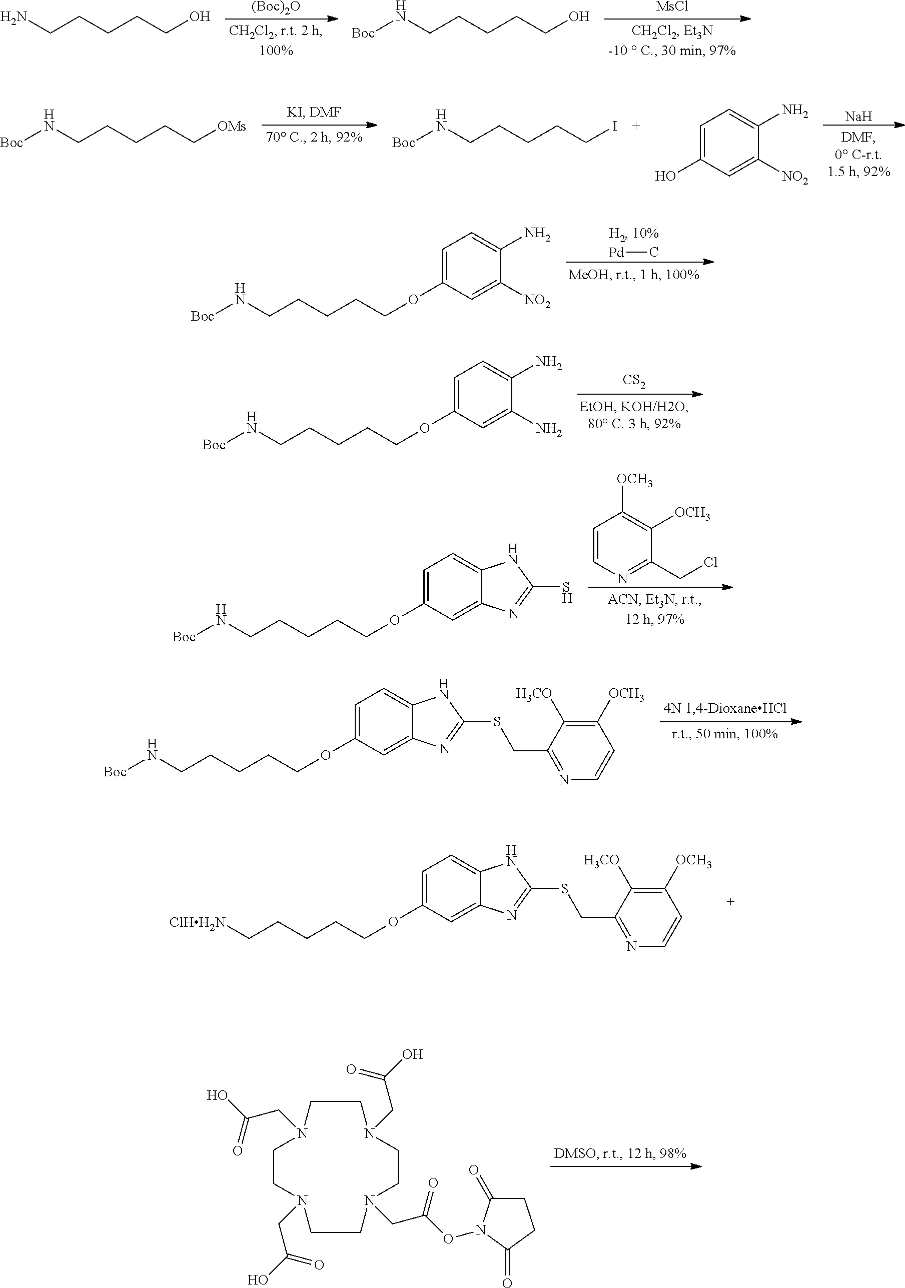

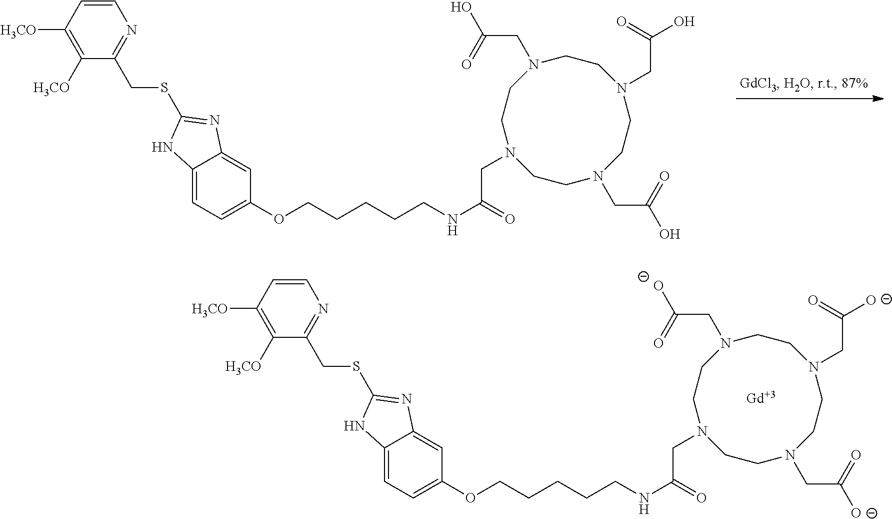

### tert-butyl 5-hydroxypentylcarbamate

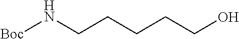

5-aminopentan-1-ol (5.0 g, 48.47 mmol) and di-tent-butyl dicarbonate (12.69 g, 58.16 mmol) were dissolved in anhydrous DCM (25 mL) and stirred under nitrogen for 2 hours, water (15 mL) was added and the product was extracted in DCM (3×20 mL). The combined organic extracts were washed with aqueous NaHCO₃, brine, dried over MgSO₄, filtered and concentrated to give the product as a colorless oil (9.85 g, 100%). ¹H NMR (CDCl₃): δ 3.62 (m, 2H), 3.11 (m, 2H), 1.62-1.31 (m, 15H). m/z (ESI) 204.20 (M⁺+1).

### 5-(tert-butoxycarbonylamino)pentyl methanesulfonate

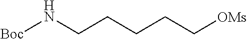

To a stirred mixture of tert-butyl 5-hydroxypentylcarbamate (9.8 g, 48.2 mmol), and triethyl amine (13.44 mL, 96.0 mmol) in DCM (85 mL) at −10 ° C. was added methanesulfonyl chloride (4.5 mL, 57.9 mmol). The resulting mixture was stirred at −10 ° C. for 3 hours. The reaction was quenched with the addition of water (5 mL). The two phases were separated and the organic phase was washed with water, brine, dried over MgSO₄, filtered and evaporated under reduced pressure to afford the title compound as a yellow oil (13.16 g, 97%). The product was used in the next step without any further purification. ¹H NMR (CDCl₃): δ 4.22 (t, 2H, J=6.42 Hz), 3.09 (m, 2H), 1.82 (m, 4H), 1.56-1.40 (m, 11H). m/z (ESI) 304.26 (M⁺+Na).

### tert-butyl 5-iodopentylcarbamate

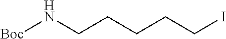

5-(tert-butoxycarbonylamino)pentyl methanesulfonate (13.0 g, 46.2 mmol) was dissolved in DMF (100 mL) and combined with potassium iodide (23.01 g, 139.0 mmol). The reaction mixture was heated at 70° C. for 2 hours and then cooled to room temperature. Water (50 mL) was added and the product was extracted into diethyl ether (3×50 mL). The combined organic extracts were washed with water, brine, dried over MgSO4, filtered and concentrated under vacuum to afford crude material. This material was purified by silica gel column chromatography using 20% ethyl acetate/hexane as eluent to afford the title compound as a pale yellow oil (13.31 g, 92%). ¹H NMR (CDCl₃): δ 3.18 (t, 2H, J=6.93 Hz), 3.11 (m, 2H), 1.83 (m, 2H), 1.59-1.44 (m, 13H). m/z (ESI) 314.08 (M⁺+1).

### tent-butyl-5-(4-amino-3-nitrophenoxy)pentylcarbamate

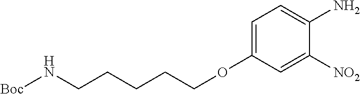

tent-butyl-5-iodopentylcarbamate (5.022 g, 16.04 mmol) and 4-hydroxy-2-nitroaniline (2.97 g, 19.24 mmol) were dissolved in dry DMF (45 mL) and the reaction mixture was cooled to 0° C. (ice-bath), NaH (2.0 g, 41.7 mmol) was added gradually and the reaction mixture was stirred under nitrogen at 0° C. for 30 minutes and then at room temperature for 1 hour. Upon completion of the reaction, water (30 mL) was added and the product was extracted into diethyl ether (3×50 mL). The combined organic extracts were washed with brine, dried over MgSO₄, filtered and evaporated under reduced pressure. The crude product was purified by flash chromatography using 20% ethyl acetate/hexane to afford the title product as a pale yellow oil (5.0 g, 92%). ¹H NMR (CDCl₃): δ 7.51 (d, 1H, J=2.89 Hz), 7.05 (dd, 1H, J=9.08 Hz, J=2.89 Hz), 6.78 (d, 1H, J=9.08 Hz), 4.59 (br s, 2H), 3.92 (t, 2H, J=6.35 Hz), 3.14 (t, 2H, J=6.56 Hz), 1.72 (m, 2H), 1.57-1.45 (m, 13H). m/z (ESI) 362.26 (M⁺+Na).

### tert-butyl 5-(3,4-diamino-phenoxy)pentylcarbamate

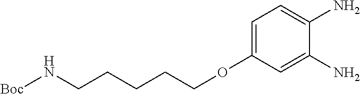

tert-butyl 5-(4-amino-3-nitrophenoxy)pentylcarbamate (5.0 g, 14.73 mmol) was dissolved in methanol (55 mL) and stirred over H₂ catalyzed by Pd 10% on carbon for 1 hour. The reaction mixture was filtered over celite and concentrated under reduced pressure, the resulting crude material was purified by flash chromatography using 5% methanol/dichloromethane to afford the title product as a brown oil (4.56 g, 100%). ¹H NMR (CDCl₃): δ 6.61 (d, 1H, J=8.21 Hz), 6.31 (d, 1H, J=2.63 Hz), 6.24 (dd, 1H, J=8.21 Hz, J=2.63 Hz), 4.54 (br s, 4H), 3.86 (t, 2H, J=6.39 Hz), 3.14 (t, 2H, J=6.56 Hz), 1.74 (m, 2H), 1.61-1.44 (m, 13H). m/z (ESI) 310.29 (M⁺+1)

### tert-butyl 5-(2-mercapto-1H-benzo [d] imidazol-5-yloxy)pentylcarbamate

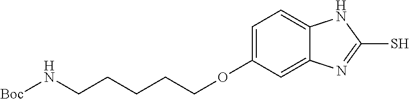

To a mixture of ethanol (60 mL), H₂O (10 mL) and KOH (0.725 g, 12.93 mmol) was added tert-butyl 5-(3,4-diamino-phenoxy)pentylcarbamate (4.0 g, 12.93 mmol) and carbon disulfide (0.984 g, 12.93 mmol). The mixture was stirred under reflux for 3 hours at 75-80° C. Upon completion (TLC monitored completion of reaction), the reaction mixture was removed from heat, charcoal (3 g) was added, and the reaction mixture was refluxed for an additional 10 minutes and then filtered over celite. Hot water (60 mL) added to the filtrate followed by acetic acid (5 mL) dissolved in water (10 mL) and cooled on an ice bath. The product was extracted with dichloromethane (3×25 mL), the combined organic extracts were washed with brine, dried over MgSO₄, filtered and concentrated under reduced pressure. The crude product was purified by silica-gel column chromatography using 5% methanol/dichloromethane as an eluent to afford the title compound as a brown-gummy solid (2.28 g, 50%). ¹H NMR (CDCl₃): δ 11.87 (s, 1H), 11.85 (s, 1H), 7.10 (d, 1H, J=8.50 Hz), 7.75 (d, 1H, J=1.62 Hz), 6.70 (dd, 1H, J=8.50 Hz, J=1.62 Hz), 4.77 (br s, 1H), 3.87 (t, 2H, J=5.92 Hz), 3.15 (br s, 2H), 1.75 (m, 2H), 1.66-1.26 (m, 13H). m/z (ESI) 352.03 (M⁻+1).

### tert-butyl 5-(2-((3,4-dimethoxypyridin-2-yl)methylthio)-1H-benzo [d]imidazol-5-yloxy)pentylcarbamate

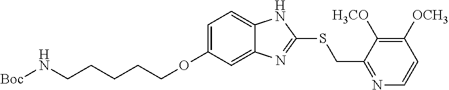

tert-butyl 5-(2-mercapto-1H-benzo[d]imidazol-5-yloxy)pentylcarbamate (0.9 g, 2.56 mmol), 2-(chloromethyl)-3,4-dimethoxypyridine. HCl (0.689 g, 3.07 mmol) and triethyl amine (1.8 mL, 12.80 mmol) were dissolved in dry acetonitrile (20 mL) and stir the reaction mixture under nitrogen at room temperature overnight (TLC monitored completion of reaction). The solvent was removed under reduced pressure and the resulting crude product was purified by silica-gel column chromatography using 2% methanol/dichloromethane as an eluent to afford the title product as a brownish solid (1.25 g, 97%). ¹H NMR (CDCl₃): δ 8.25 (d, 1H, J=5.62 Hz), 7.40 (d, 1H, J=8.70 Hz), 7.02 (d, 1H, J=1.67 Hz), 6.84 (d, 1H, J=5.62 Hz), 6.80 (dd, 1H, J=8.70 Hz, J=1.67 Hz), 4.56 (br s, 1H), 4.37 (s, 2H), 3.99 (t, 2H, J=6.36 Hz), 3.95 (s, 3H), 3.93 (s, 3H), 3.15 (t, 2H, J=5.98 Hz), 1.83 (m, 2H), 1.55-1.44 (m, 13H). m/z (ESI) 503.25 (M⁺+1)

### 5-(2-((3,4-dimethoxypyridin-2-yl)methylthio)-1H-benzo [d]imidazol-5-yloxy)pentan-1-amine-HCl

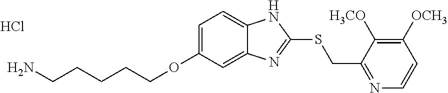

tert-butyl 5-(2-((3,4-dimethoxypyridin-2-yl)methylthio)-1H-benzo[d]imidazol-5-yloxy)pentylcarbamate (1.20 g, 2.387 mmol) was dissolved in 4M HCl-dioxane (30 mL) and stirred under nitrogen for 50 minutes. The solvent was evaporated under reduced pressure and the residue was washed with minimal cold ether to afford the target compound as a brown powder (1.048 g, 100%). ¹H NMR (DMSO-d₆): δ 8.53 (d, 1H, J=6.58 Hz), 8.12 (s, 3H), 7.59 (d, 1H, J=6.58 Hz),7.55 (d, 1H, J=8.89 Hz), 7.14 (d, 1H, J=2.04 Hz), 7.08 (dd, 1H, J=8.89 Hz, J=2.04 Hz), 4.97 (s, 2H), 4.06 (s, 3H), 4.01 (t, 2H, J=6.31 Hz), 3.81 (s, 3H), 2.77 (m, 2H), 1.75 (m, 2H), 1.69 (m, 2H), 1.51 (m, 2H). m/z (ESI) 403.18 (M⁺+1).

### 2,2′,2″-(10-(2-(5-(2-((3,4-dimethoxypyridin-2-yl)methylthio)-1H-benzo[d]imidazol-5-yloxy)pentylamino)-2-oxoethyl)-1,4,7,10-tetraazacyclododecane-1,4,7-triyl)triacetic acid

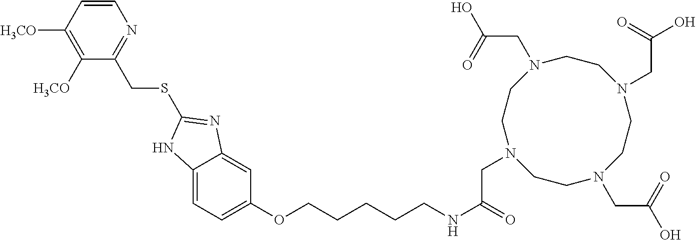

To a solution of 5-(2-((3,4-dimethoxypyridin-2-yl)methylthio)-1H-benzo[d]imidazol-5-yloxy)pentan-1-amine. HCl (50.0 mg, 0.110 mmol) in dry DMSO (0.85 mL) was added triethylamine (0.613 mL, 4.40 mmol). The mixture was stirred until dissolved under nitrogen. 2,2′,2″-(10-(2-(2,5-dioxopyrrolidin-1-yloxy)-2-oxoethyl)-1,4,7,10-tetraazacyclododecane-1,4,7-triyl)triacetic acid (0.109 mg, 0.110 mmol) was added to the reaction mixture and stirred for 12 hours at room temperature under nitrogen (ESI monitored completion of reaction). Upon completion of the reaction, the solvent was evaporated using a lypholizer and the crude product was purified by HPLC to afford a yellowish thick oil (87.0 mg, 98%). HPLC: 10% B-100% B in 40 min (B=80% aq. CH₃CN with 0.1% TFA, A=H₂O with 0.1% TFA); FR=8 mL/min; λₘₐₓ=254 nm. RT=19.4 min. ¹H NMR (DMSO-d₆): δ 8.56 (br s, 1H), 8.41 (d, 1H, J=6.21 Hz), 7.59 (d, 1H, J=8.89 Hz),7.41 (d, 1H, J=6.21 Hz), 7.13 (s, 1H), 6.98 (dd, 1H, J=8.89 Hz, J=1.91 Hz), 4.78 (s, 2H), 4.01 (s, 3H), 4.01 (br s, 2H), 3.83 (s, 3H), 3.62-3.13 (m, 18H), 1.76 (m, 2H), 1.49 (m, 4H). m/z (ESI) 789.15 (M⁺+1)

### monogadolinium(II) mono(2,2’,2″-(10-(2-(5-(2-((3,4-dimethoxypyridin-2-yl)methyl-thio)-1H-benzo[d]imidazol-5-yloxy)pentylamino)-2-oxoethyl)-1,4,7,10-tetraazacyclo-dodecane-1,4,7-triyl)triacetate)

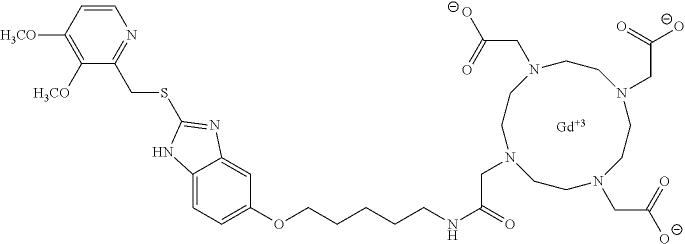

To an aqueous solution (0.25 mL) of 2,2′,2″-(10-(2-(5-(2-((3,4-dimethoxypyridin-2-yl)methylthio)-1H-benzo[d]imidazol-5-yloxy)pentylamino)-2-oxoethyl)-1,4,7,10-tetraazacyclododecane-1,4,7-triyl)triacetic acid (50.0 mg, 0.063 mmol) was added GdCl₃ (17.0 mg, 0.063 mg) dissolved in water (0.25 mL). After the 90% addition of GdCl₃, the pH of the reaction mixture was adjusted to between 5 and 6 with an aqueous solution of 0.01 N NaOH and then the remaining GdCl₃ solution was added drop wise at approximately pH 6. After the complete addition of the GdCl₃ solution, the pH of the reaction mixture was then adjusted to between 7 and 8 with an aqueous solution of 0.01 N NaOH, and if necessary a solution of 0.01 N HCl was used to adjust the pH. The reaction mixture was then passed through millipore filter, and purified using HPLC to afford a clear thick oil (52.0 mg, 87%). HPLC: 10% B-100% B in 40 min (B=80% aq. CH₃CN with 0.1% TFA, A=H₂O with 0.1% TFA); FR=8 mL/min; λ=254 nm. RT=19.0. m/z (ESI) 944.24 (M⁺+1)

### Example 2

### Preparation of 2-(chloromethyl)-3,4-dimethoxypyridine hydrochloride

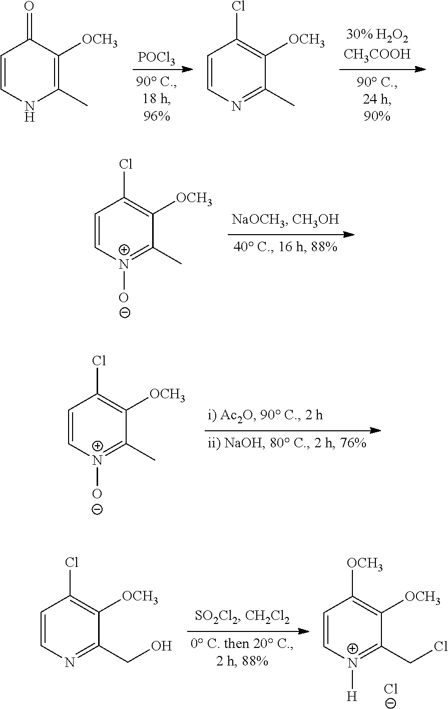

### 4-Chloro-3-methoxy-2-methylpyridine

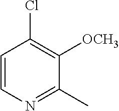

3-Methoxy-2-methyl-4(1H)-pyridone (27.8 g, 0.2 mol) was added to phosphorus oxychloride (200 mL) and the resulting mixture was stirred at 90° C. for 18 hours under nitrogen. The solution was then concentrated under reduced pressure and cooled to 20° C. The residue was treated with ice-water and the pH was adjusted using 40% sodium hydroxide to pH 12, and extracted with dichloromethane (3×100 mL). The combined organic extracts were distilled at reduced pressure to give the title product (30.2 g, 96%). ¹H NMR (CDCl₃): δ 8.14 (d, 1H, J=5.32 Hz), 7.17 (d, 1H, J=5.10 Hz), 3.85 (s, 3H), 2.54 (s, 3H). m/z (ESI) 157.91 (M⁺+1).

### 4-Chloro-3-methoxy-2-methylpyridine N-Oxide

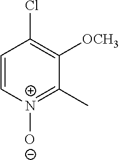

A mixture of 4-chloro-3-methoxy-2-methylpyridine (13.5 g, 85.0 mmol), acetic acid (250 mL), and hydrogen peroxide (33.5 mL of 30% solution, 0.34 mol) was heated to 90° C. for 24 hours. Upon completion, the solution was evaporated in vacuo to afford a crude oil which was purified by silica gel column chromatography using dichloromethane/methanol) to afford the title product (13.25 g, 90%). ¹H NMR (CDCl₃): δ 8.22 (d, 1H, J=7.05 Hz), 7.26 (d, 1H, J=7.05 Hz), 3.90 (s, 3H), 2.55 (s, 3H). m/z (ESI) 196.12 (M+Na).

### 3,4-dimethoxy-2-methylpyridine-N-oxide

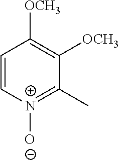

A mixture of 4-chloro-3-methoxy-2-methylpyridine-N-oxide, (9.0 g, 50.0 mmol) and sodium methoxide (5.4 g, 100.0 mmol) in dry methanol (150 mL) was stirred at 40° C. for 16 hours. After cooling, the solution was adjusted to pH 7 by addition of concentrated sulfuric acid. The mixture was evaporated in vacuo and the residue extracted with toluene (100 mL). After filtration, to remove insoluble inorganic salts, the filtrate was evaporated in vacuo to afford a yellow oil. Chromatography (silica gel, dichloromethane/methanol) followed by trituration with petroleum ether at 40° C. afforded the title compound (10.4 g, 88%). ¹H NMR (CDCl₃): δ 8.09 (d, 1 H, J=7.28 Hz), 6.71 (d, 1H, J=7.28 Hz), 3.92 (s, 3H), 3.85 (s, 3H), 2.50 (s, 3H). m/z (ESI) 170.19 (M⁻+1).

### hydroxymethyl-3,4-dimethoxypyridine

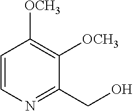

3,4-dimethoxy-2-methylpyridine-N-oxide (9.6 g, 56.8 mmol) dissolved in acetic anhydride (50 mL) was heated at 90° C. for 2 hours. After evaporation in vacuo, the dark oily residue was agitated with 2N NaOH (40 mL) for 2 hours at 80° C. After cooling, the product was extracted into dichloromethane (3×50 mL), dried over K₂CO₃, and concentrated in vacuo to a volume of approximately 10 mL. Addition of petroleum ether afforded the product as a colorless solid (7.20 g, 76%). ¹H NMR (CDCl₃): δ 8.22 (d, 1H, J=5.6 Hz), 6.82 (d, 1H, J=5.6 Hz), 4.76 (s, 2H), 3.93 (s, 3H), 3.85 (s, 3H). m/z (ESI) 192.15 (M⁻+Na).

### 2-(chloromethyl)-3,4-dimethoxypyridine hydrochloride

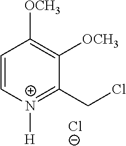

Thionyl chloride (4 mL, 15.0 mmol) in dry dichloromethane(20 mL) was added drop wise to a cooled (0-5° C.) stirred solution of hydroxymethyl-3,4-dimethoxypyridine (6.76 g, 40.0 mmol) in dichloromethane (60 mL). The mixture was allowed to warm up to 20° C. and, after 2 hours, concentrated to low volume in vacuo. Addition of toluene afforded the title product as a colorless solid (8.4 g, 93%). ¹H NMR (CDCl₃): δ 8.56 (d, 1H, J=6.63 Hz), 7.56 (d, 1H, J=6.63 Hz), 5.06 (s, 2H), 4.23 (s, 3H), 4.09 (s, 3H), m/z (ESI) 187.93 (M⁻+1).

### Example 3

### Stability of (Gd-DOTA-3COOH-S-Pantoprazole)

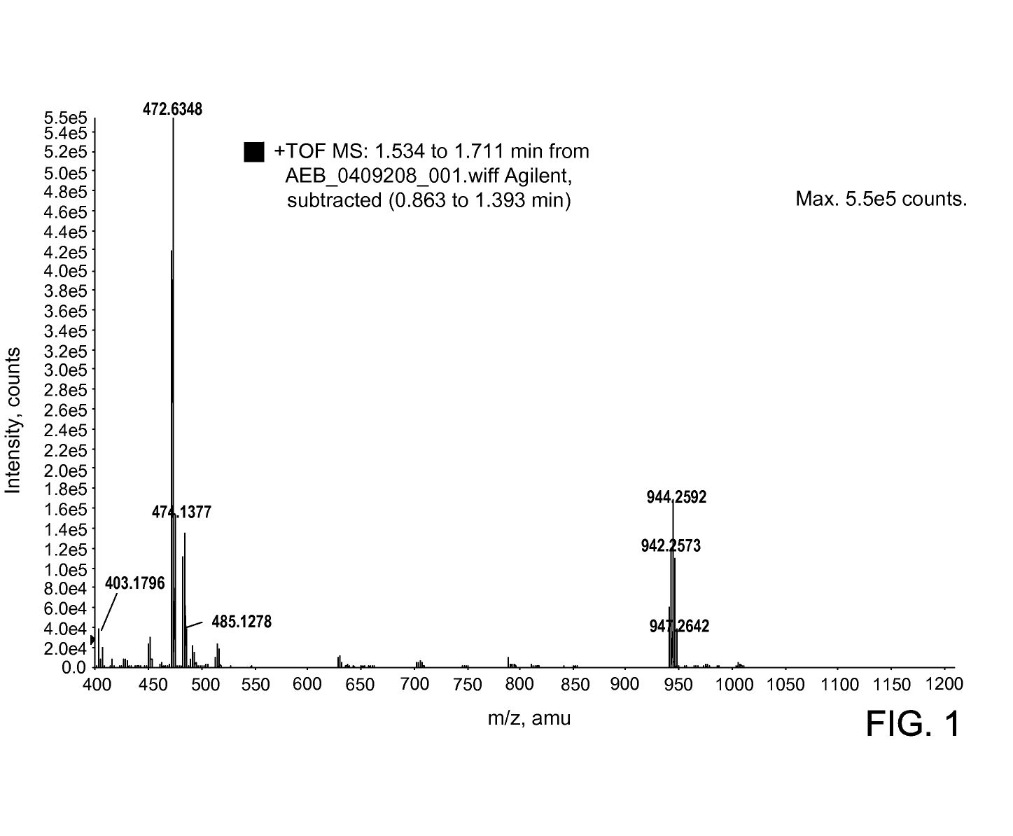

A contrast agent was prepared as described in Examples 1 and 2. The stability of the complex was confirmed by HPLC following storage of approximately 6 months after preparation. As shown in FIG. 1, two main ions are noted, both from the sulfide, with two different charges (960, 480.6). Similar results were obtained by HPLC on this same sample approximately 1 year after preparation.

### Example 4

### Imaging Studies

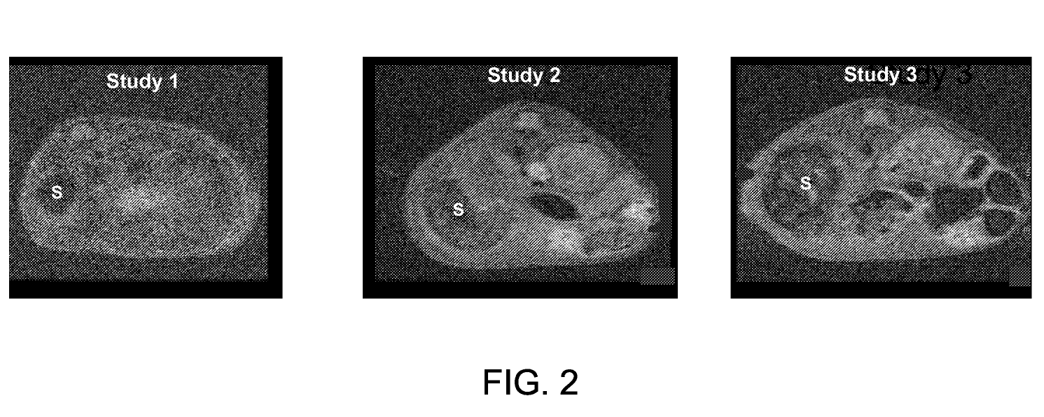

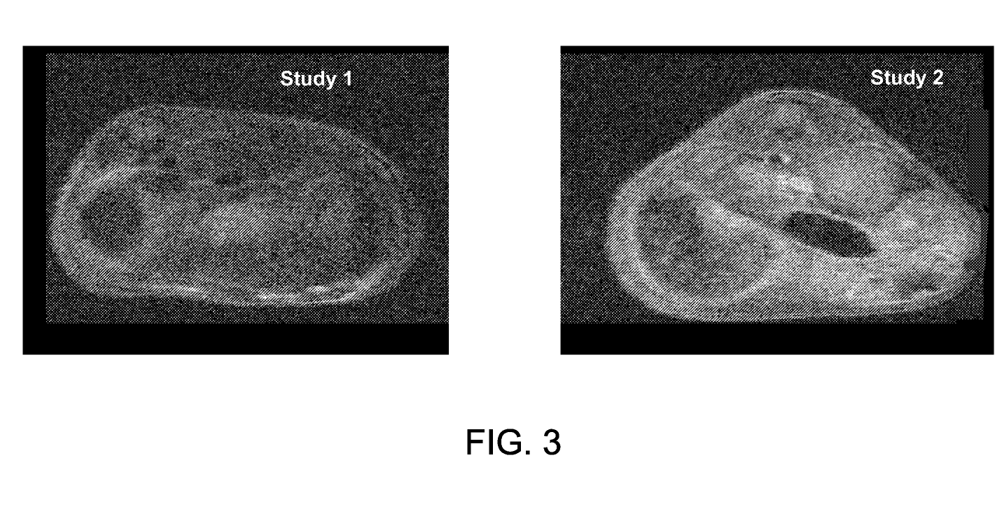

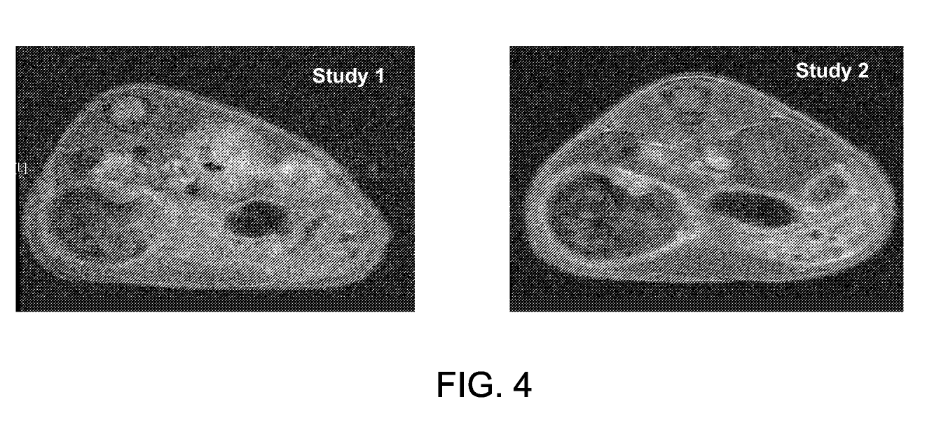

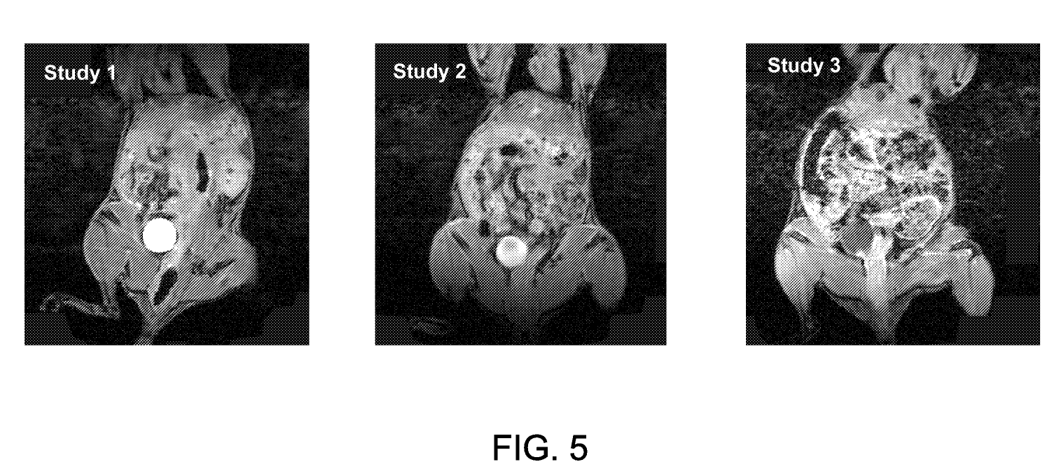

Three MRI studies were performed with the commercially available gadolinium agent (Magnevist) and two studies were performed with Gd-DOTA-3COOH-S-Pantoprazole in mice. Images were acquired under fasting and postprandial conditions (i.e., after feeding with 80 μL egg yolk). Mice were administered 2.5 mg of Magnevist or Gd-DOTA-3COOH-S-Pantoprazole in a volume of 10 μL intravenously. As shown in FIG. 2, under fasting conditions, MRI without intravenous contrast suggests that the stomach wall blends imperceptibly with the surrounding tissue. In the figure, the gastric lumen is marked by an “s”. In FIGS. 3 and 4, the images from study 2 indicate that the PPI-gadolinium agent improves signal in the gastric wall under fasting (FIG. 3) and posprandial conditions (FIG. 4). As shown in FIG. 3, the signal in the gastric wall is improved in Study 2 versus Study 1. Moreover, the gastric wall signal is enhanced in post versus pre contrast scans. In addition, the signal in the gastric wall was sustained for the duration of imaging (i.e. 1.5 hours) suggesting that the complex binds effectively to the stomach wall. As shown in FIG. 4, again, the signal in the gastric wall was improved in Study 2 versus Study 1 under postprandial conditions.

As shown in FIG. 4, post-contrast urinary excretion (i.e. bladder signal) was lower with the PPI-gadolinium agent than with Mangevist, which indicates that the contrast agent binds effectively to the gastric wall. Moreover, the PPI-gadolinium agent also labels the colonic wall, which also contains proton pumps, to a greater extent than Magnevist. Thus, the PPI-gadolinium conjugate may also improve visualization of the colonic wall.

### Example 5

### Imaging Studies

Ten abdominal MRI studies in mice were performed under three conditions in sequential order: fasting pre-contrast, fasting post-contrast, and postprandial post-contrast (after feeding with 80 μL egg yolk). Five studies each were conducted with a commercially-available gadolinium contrast agent (gadopentetic acid, Magnevist™) and with Gd-DOTA-3COOH-S-Pantoprazole. Mice were administered 2.5 mg of Magnevist or Gd-DOTA-3COOH-S-Pantoprazole in a volume of 10 μL intravenously.

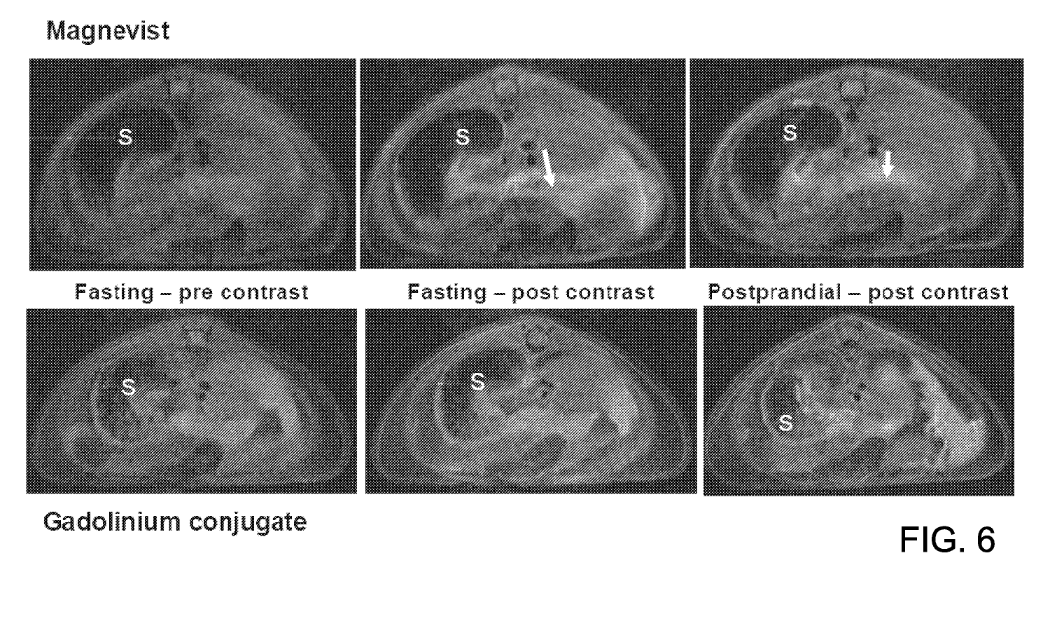

As shown in FIG. 6, gastric MRI using Gd-DOTA-3COOH-S-Pantoprazole increases gastric wall signal and enables automated segmentation when compared to no contrast agent or to Magnevist alone. In these images the gastric lumen is marked “s.” The images were analyzed by standard techniques (i.e., full-width-half-maximum method). Following administration of Magnevist, the uptake signal increased diffusely, as shown by the arrow. The automated segmentation algorithm, however, could not accurately identify the gastric outline since the stomach wall detected by the program (shown by the white box) is outside the stomach. In the Gd-DOTA-3COOH-S-Pantoprazole study, the algorithm only identified the stomach boundary after contrast, i.e., fasting and postprandial images, but the conjugate was capable of discriminating the stomach wall from its contents and surrounding tissues.

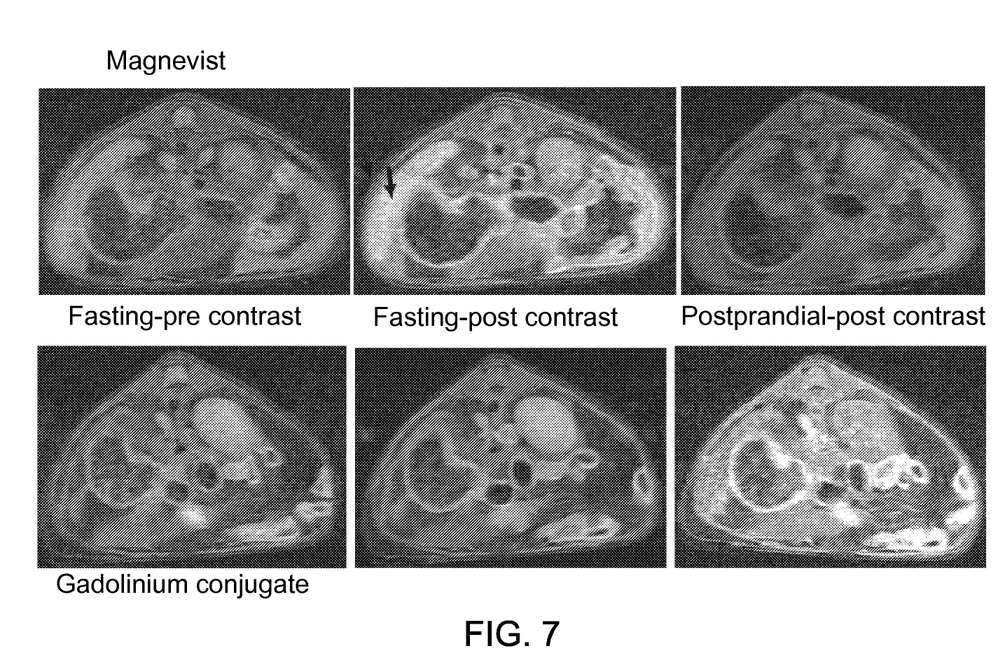

The images shown in FIG. 7 compare the splenic uptake exhibited with Magnevist and Gd-DOTA-3COOH-S-Pantoprazole. The upper (center) panel in FIG. 7 shows marked splenic uptake (as indicated by the white arrow) following administration of Magnevist; consequently, the boundary between the stomach and spleen is blurred and the algorithm overestimates stomach wall thickness (white box). In comparison, the gastro-splenic boundary is identifiable after Gd-DOTA-3COOH-S-Pantoprazole administration (lower panel) and the signal intensity in the stomach and bowel loops increased between the fasting and postprandial post-contrast images. These images illustrate the ability of the Gd-DOTA-3COOH-S-Pantoprazole contrast agent to discriminate the gastric wall from the surrounding tissues during gastric MRI.

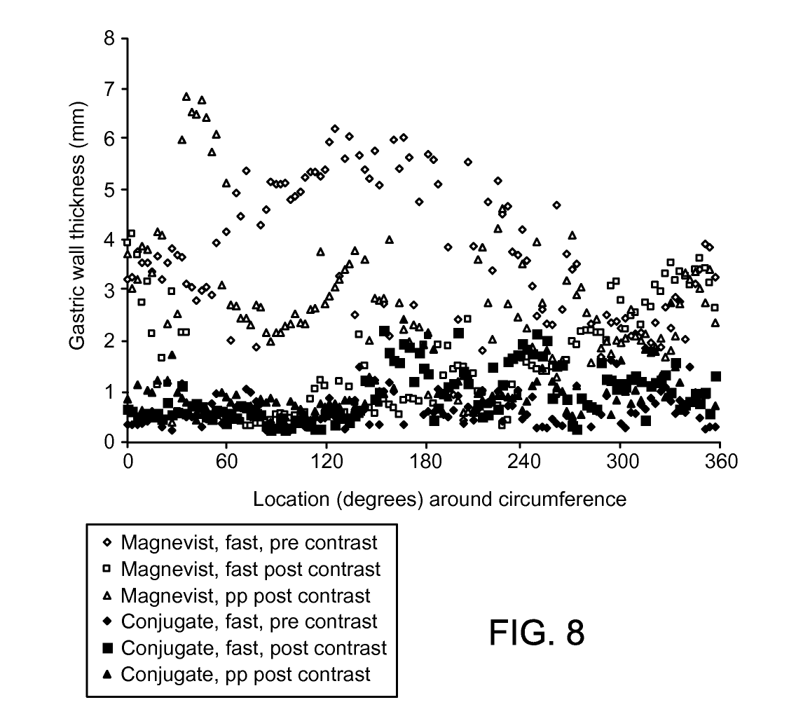

Using the data shown in FIG. 7, a series of wall thickness measurements were made (see FIG. 8). For the reasons highlighted above, the wall thickness measurements were accurate and tightly distributed after imaging with Gd-DOTA-3COOH-S-Pantoprazole (see FIG. 8, filled symbols) compared to Magnevist (see FIG. 8, open symbols).

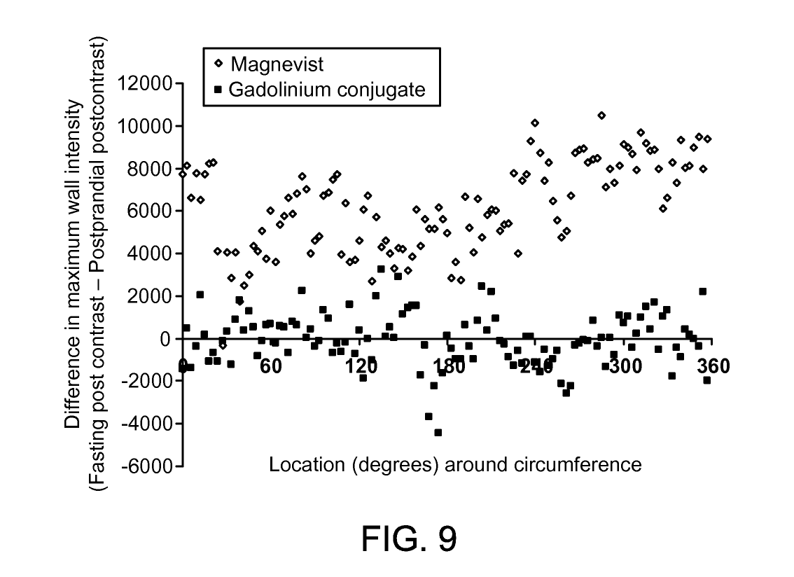

Comparison of gastric wall signal intensity over time after intravenous Magnevist and Gd-DOTA-3COOH-S-Pantoprazole is shown in FIG. 9. Data were sampled at 120 equidistant points around the gastric wall. The differences between fasting post contrast and postprandial post contrast images following administration of Magnevist show a higher difference than those following administration of Gd-DOTA-3COOH-S-Pantoprazole. This suggests that Gd-DOTA-3COOH-S-Pantoprazole was retained in the gastric wall for a longer duration, which is consistent with binding to the gastric acid pumps, compared to Magnevist.

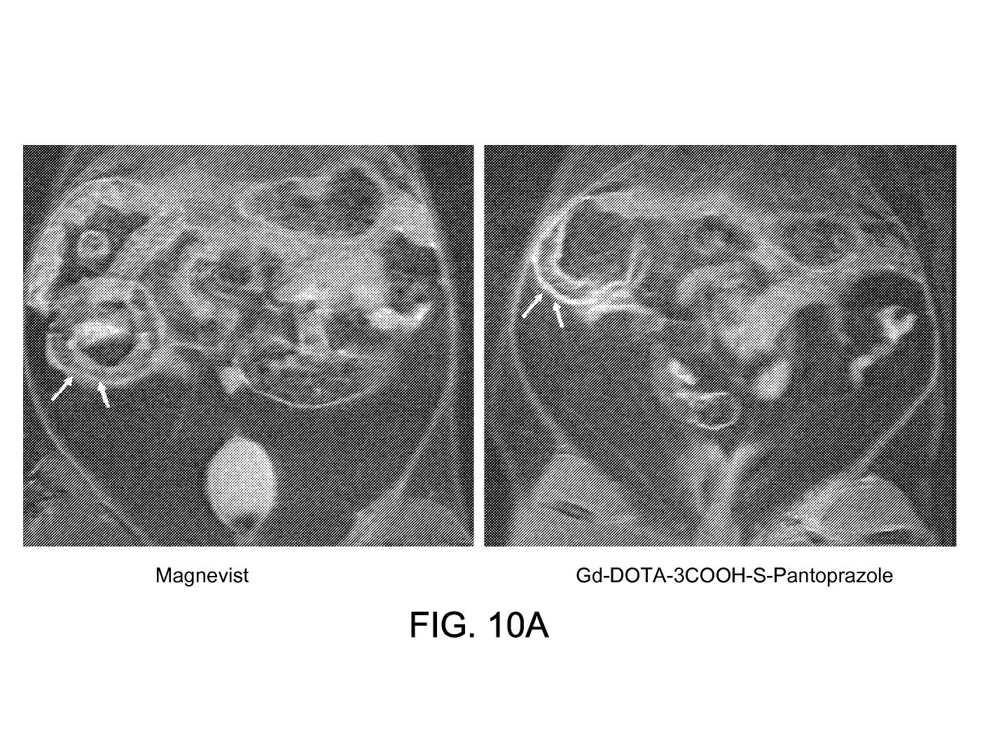

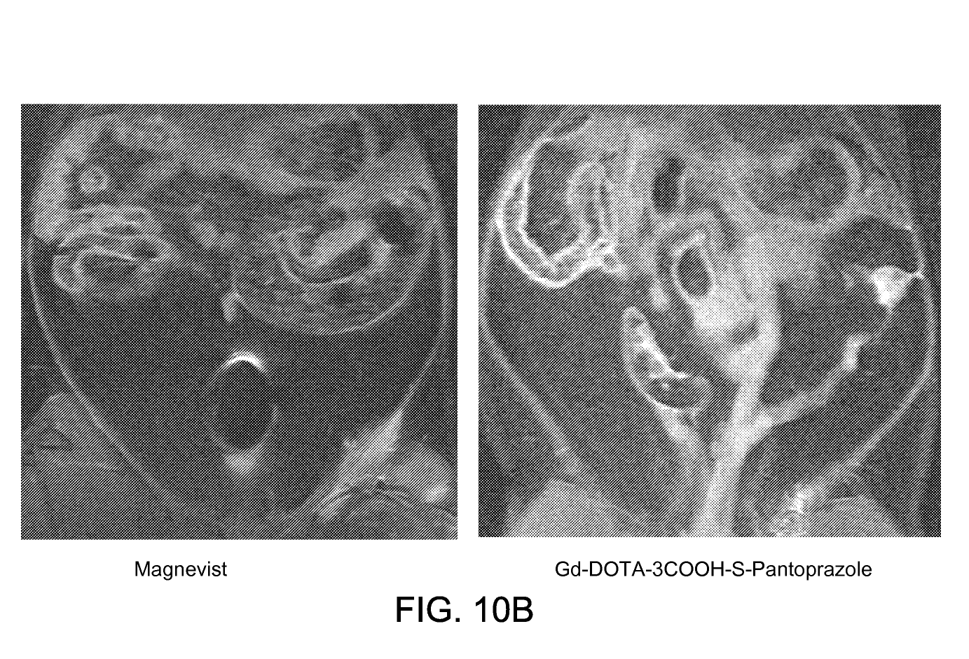

Comparison of colonic wall signal after administration of Magnevist (left panel) and Gd-DOTA-3COOH-S-Pantoprazole (right panel) is shown in FIG. 10a. Increased colonic signal (arrows) is observed after administration of Gd-DOTA-3COOH-S-Pantoprazole compared to Magnevist alone, which is consistent with the distribution of H⁺-K⁺ ATPase in the colon. FIG. 10b shows the colonic postprandial post contract images. Accordingly, Gd-DOTA-3COOH-S-Pantoprazole enables identification of the stomach and colon by MRI.

The data described in this experiment demonstrates that Gd-DOTA-3COOH-S-Pantoprazole enables semi-automated segmentation imaging of the stomach and colon compared to Magnevist alone.

A number of embodiments of the invention have been described. Nevertheless, it will be understood that various modifications may be made without departing from the spirit and scope of the invention. Accordingly, other embodiments are within the scope of the following claims.

What is claimed is:

1. A contrast agent comprising a compound of formula I:
[PPI]ₙ−[L]ₘ−[C]ₚ
wherein:
PPI is a proton pump inhibitor;
L is a linker;
C is a physiologically compatible metal chelating group;
n is an integer from one to five;
m is an integer from zero to ten; and
p is an integer from one to ten;
or a pharmaceutically acceptable salt thereof

2. The contrast agent of claim 1, wherein the PPI is selected from the group consisting of: omeprazole, lansoprazole, dexlansoprazole, esomeprazole, pantoprazole, and rabeprazole.

3. The contrast agent of claim 1, wherein the PPI comprises a compound of formula II: 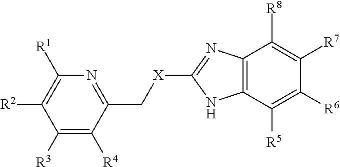
wherein:
X is S or S═O;
R¹, R², R³, R⁴, R⁵, R⁶, R⁷, and R⁸ are independently selected from H, C₁₋₁₀ alkyl, C₂₋₁₀ alkenyl, C₂₋₁₀ alkynyl, or OR⁹, NR⁹, or SR⁹, wherein at least one of R¹-R⁸ is OR⁹;
R⁹ is independently selected from H, C₁₋₁₀ alkyl, C₂₋₁₀ alkenyl, C₂₋₁₀ alkynyl, or a linkage site to L, if present, or C, wherein at least one R⁹ is a linkage site;
or a pharmaceutically acceptable salt thereof

4. The contrast agent of claim 3, wherein the compound of formula II is: 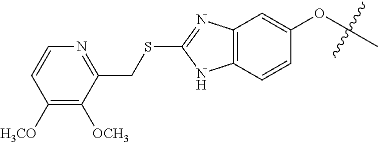
or a pharmaceutically acceptable salt thereof

5. The contrast agent of claim 3, wherein the compound of formula II is: 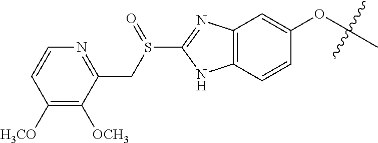
or a pharmaceutically acceptable salt thereof

6. The contrast agent of claim 1, wherein C is complexed to a paramagnetic metal ion.

7. The contrast agent of claim 6, wherein the paramagnetic metal ion is selected from the group consisting of: Gd(III), Fe(III), Mn(II), Mn(III), Cr(III), Cu(II), Dy(III), Ho(III), Er(III), Pr(III), Eu(II), Eu(III), Tb(III), and Tb(IV).

8. The contrast agent of claim 7, wherein the paramagnetic metal ion is Gd(III).

9. The contrast agent of claim 1, wherein the physiologically compatible metal chelating group (C) comprises a cyclic or an acyclic organic chelating agent.

10. The contrast agent of claim 9, wherein the cyclic or acyclic organic chelating agent is selected from the group consisting of DTPA, DOTA, HP-DO3A, NOTA, DOTAGA, Glu-DTPA, and DTPA-BMA.

11. The contrast agent of claim 10, wherein the cyclic or acyclic organic chelating agent comprises DTPA, DOTAGA, and DOTA.

12. The contrast agent of claim 1, wherein the compound of formula I is: 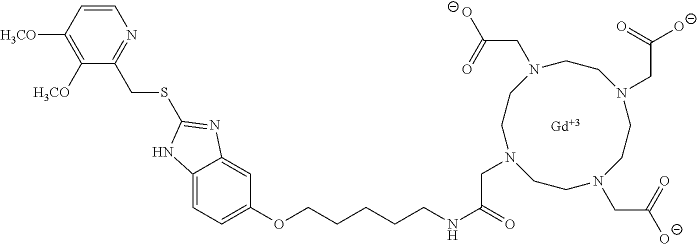
or a pharmaceutically acceptable salt thereof.

13. The contrast agent of claim 1, wherein the compound of formula I is: 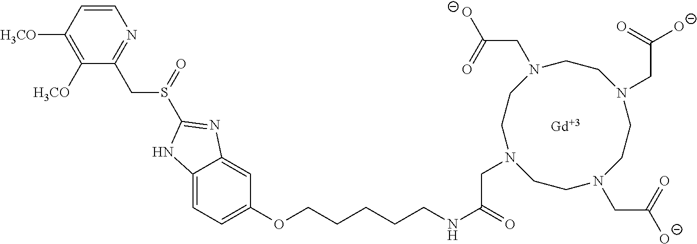
or a pharmaceutically acceptable salt thereof.

14. A pharmaceutical composition comprising a contrast agent comprising a compound of formula I:
[PPI]ₙ−[L]ₘ−[C]ₚ
wherein:
PPI is a proton pump inhibitor;
L is a linker;
C is a physiologically compatible metal chelating group;
n is an integer from one to five; and
m is an integer from zero to ten; and
p is an integer from one to ten;
or a pharmaceutically acceptable salt thereof; and
a pharmaceutically acceptable carrier, adjuvant or vehicle.

15. A method of MRI imaging, the method comprising:
(a) administering to a subject an effective amount of a contrast agent comprising a compound of formula I:
[PPI]ₙ−[L]ₘ−[C]ₚ
wherein:
PPI is a proton pump inhibitor;
L is a linker;
C is a physiologically compatible metal chelating group;
n is an integer from one to five; and
m is an integer from zero to ten; and
p is an integer from one to ten;
or a pharmaceutically acceptable salt thereof; and
(b) performing MRI imaging on the subject.

16. A method of imaging the stomach of a subject, the method comprising:
(a) administering to the subject an effective amount of a contrast agent comprising a compound of formula I:
[PPI]ₙ−[L]ₘ−[C]ₚ
wherein:
PPI is a proton pump inhibitor;
L is a linker;
C is a physiologically compatible metal chelating group;
n is an integer from one to five; and
m is an integer from zero to ten; and
p is an integer from one to ten;
or a pharmaceutically acceptable salt thereof; and
(b) performing MRI imaging of the stomach.

17. The method of claim 16, wherein the imaging of the stomach comprises imaging of the stomach wall.

18. The method of claim 16, wherein the imaging of the stomach comprises imaging of the stomach contents.

19. The method of claim 16, wherein the imaging of the stomach comprises imaging of the stomach wall and contents simultaneously.

20. A method of imaging the stomach wall of a subject, the method comprising:
(a) administering to the subject an effective amount of a contrast agent comprising a compound of formula I:
[PPI]ₙ−[L]ₘ−[C]ₚ
wherein:
PPI is a proton pump inhibitor;
L is a linker;
C is a physiologically compatible metal chelating group;
n is an integer from one to five; and
m is an integer from zero to ten; and
p is an integer from one to ten;
or a pharmaceutically acceptable salt thereof; and
(b) performing MRI imaging of the stomach wall.

21. A method of imaging the stomach contents of a subject, the method comprising:
(a) administering to the subject an effective amount of a contrast agent comprising a compound of formula I:
[PPI]ₙ−[L]ₘ−[C]ₚ
wherein:
PPI is a proton pump inhibitor;
L is a linker;
C is a physiologically compatible metal chelating group;
n is an integer from one to five; and
m is an integer from zero to ten; and
p is an integer from one to ten;
or a pharmaceutically acceptable salt thereof; and
(b) performing MRI imaging of the stomach contents.

22. A method of imaging the stomach wall and stomach contents of a subject simultaneously, the method comprising:
(a) administering to the subject an effective amount of a contrast agent comprising a compound of formula I:
[PPI]ₙ−[L]ₘ−[C]ₚ
wherein:
PPI is a proton pump inhibitor;
Lisa linker;
C is a physiologically compatible metal chelating group;
n is an integer from one to five; and
m is an integer from zero to ten; and
p is an integer from one to ten;
or a pharmaceutically acceptable salt thereof; and
(b) performing MRI imaging of the stomach wall and stomach contents.

23. A method of imaging stomach volume of a subject, the method comprising:
(a) administering to the subject an effective amount of a contrast agent comprising a compound of formula I:
[PPI]ₙ−[L]ₘ−[C]ₚ
wherein:
PPI is a proton pump inhibitor;
L is a linker;
C is a physiologically compatible metal chelating group;
n is an integer from one to five; and
m is an integer from zero to ten; and
p is an integer from one to ten;
or a pharmaceutically acceptable salt thereof; and
(b) performing MRI imaging of the stomach contents.

24. A method of imaging stomach motility of a subject, the method comprising:
(a) administering to the subject an effective amount of a contrast agent comprising a compound of formula I:
[PPI]ₙ−[L]ₘ−[C]ₚ
wherein:
PPI is a proton pump inhibitor;
L is a linker;
C is a physiologically compatible metal chelating group;
n is an integer from one to five; and
m is an integer from zero to ten; and
p is an integer from one to ten;
or a pharmaceutically acceptable salt thereof; and
(b) performing MRI imaging of the stomach wall.

25. A method of imaging stomach volume and motility of a subject, the method comprising:
(a) administering to the subject an effective amount of a contrast agent comprising a compound of formula I:
[PPI]ₙ−[L]ₘ−[C]ₚ
wherein:
PPI is a proton pump inhibitor;
L is a linker;
C is a physiologically compatible metal chelating group;
n is an integer from one to five; and
m is an integer from zero to ten; and
p is an integer from one to ten;
or a pharmaceutically acceptable salt thereof; and
(b) performing MRI imaging of the stomach.

26. The method of claim 25, wherein the imaging of the stomach comprises imaging of the stomach wall.

27. The method of claim 25, wherein the imaging of the stomach comprises imaging of the stomach contents.

28. The method of claim 25, wherein the imaging of the stomach comprises imaging of the stomach wall and contents simultaneously.

29. The method of claim 25, wherein the MRI imaging of the stomach is performed using a single MRI sequence.

30. The method of claim 25, wherein the subject is a human.

31. A method of imaging the colon of a subject, the method comprising:
(a) administering to the subject an effective amount of a contrast agent comprising a compound of formula I:
[PPI]ₙ−[L]ₘ−[C]ₚ
wherein:
PPI is a proton pump inhibitor;
L is a linker;
C is a physiologically compatible metal chelating group;
n is an integer from one to five; and
m is an integer from zero to ten; and
p is an integer from one to ten;
or a pharmaceutically acceptable salt thereof; and
(b) performing MRI imaging of the colon.

32. The method of claim 31, wherein the imaging of the colon comprises imaging of the colon wall.

33. The method of claim 31, wherein the imaging of the colon comprises imaging of the colon contents.

34. The method of claim 31, wherein the imaging of the colon comprises imaging of the colon wall and contents simultaneously.

35. A method of imaging viscera responsive to proton pump inhibitors in a subject, the method comprising:
(a) administering to the subject an effective amount of a contrast agent comprising a compound of formula I:
[PPI]ₙ−[L]ₘ−[C]ₚ
wherein:
PPI is a proton pump inhibitor;
L is a linker;
C is a physiologically compatible metal chelating group;
n is an integer from one to five; and
m is an integer from zero to ten; and
p is an integer from one to ten;
or a pharmaceutically acceptable salt thereof; and
(b) performing MRI imaging of the viscera.

36. The method of claim 35, wherein the imaging of the viscera comprises imaging the wall of the viscera.

37. The method of claim 35, wherein the imaging of the viscera comprises imaging of the contents of the viscera.

38. The method of claim 35, wherein the imaging of the viscera comprises imaging of the wall and the contents of the viscera simultaneously.

39. The method of claim 35, wherein the viscera is selected from one or more of the stomach, colon, kidneys, intestine, liver, and bladder.
